(19) United States
(12) Patent Application Publication
(10) Pub. No.: US 2023/0330305 A1
(43) Pub. Date: Oct. 19, 2023

(54) HYDROGEL COMPRISING A CROSS-LINKED AND SILYLATED POLYSACCHARIDE AND PROCESS FOR OBTAINING SAME

(71) Applicant: TEOXANE SA, GENEVE (CH)

(72) Inventors: Julien IEHL, PREVESSIN-MOENS (FR); Jimmy FAIVRE, VALSERHÔNE (FR)

(73) Assignee: TEOXANE SA, GENEVE (CH)

(21) Appl. No.: 18/025,317

(22) PCT Filed: Sep. 9, 2021
(86) PCT No.: PCT/IB2021/000777

§ 371 Date: Mar. 8, 2023

(30) Foreign Application Priority Data
Sep. 9, 2020 (FR) 2009135

(51) Int. Cl. A61L 27/52; A61L 27/20; A61L 27/54

(52) U.S. Cl. CPC A61L 27/52; A61L 27/20; A61L 27/54; A61L 2300/204; A61L 2430/34; A61L 2300/402

(57) ABSTRACT

The present invention relates to a process for preparing a hydrogel, comprising the following steps: a) provision of a polysaccharide or a salt thereof; b) crosslinking of the polysaccharide in the presence of 0.05 to 10 mol %, preferentially 0.1 to 2 mol %, of at least one crosslinking agent, or a salt thereof, per 1 mol of repeat units of the polysaccharide; c) functionalisation of the polysaccharide with at least one silylated molecule of formula Chem. I or a salt thereof; d) sol-gel reaction of at least one part of the Si—OR¹⁰ groups and optionally at least one part of the SiOR⁴ groups of the molecule of formula Chem. I or a salt thereof when they are present.

### TECHNICAL FIELD

The present invention relates to a process for preparing a hydrogel comprising a cross-linked polysaccharide, in particular, a process for preparing an injectable hydrogel comprising cross-linked hyaluronic acid. The present invention also relates to a hydrogel, preferably injectable, obtainable by the process, a composition comprising the hydrogel, and the uses of this hydrogel.

### PRIOR ART

Polysaccharide hydrogels are used in various fields such as aesthetic, cosmetic and therapeutic fields. In particular, they can replace biological tissues. In particular, hyaluronic acid (HA) gels find applications in ophthalmology, periodontology, rheumatology or else cosmetic surgery. Hyaluronic acid hydrogels are used in particular to fill soft tissues, preferably the skin, having volume defects such as wrinkles, scars or to increase the volume of soft tissues.

To obtain hyaluronic acid gels with desirable mechanical properties, in vivo durability and resistance to degradation for soft tissue filling, hyaluronic acid is generally cross-linked with one or more cross-linking agent(s). Conventional cross-linking agents have at least two functions reactive with functional groups present on the polysaccharide which enable them to bond polysaccharide molecules together and therefore to cross-link them. As a result, these cross-linking agents have a certain toxicity in vivo because their reactive functions with functional groups present on the polysaccharide can allow them to also react with biopolymers such as peptides, carbohydrates and DNA and therefore to cross-link them.

For questions of biocompatibility and safety of the products, it is therefore desirable to reduce the amounts of cross-linking agent conventionally used in order to preserve the least modified polysaccharide possible. Nevertheless, below a certain threshold, the gels prepared no longer have suitable properties. In particular, gels of hyaluronic acid cross-linked with 1,4-butanediol diglycidyl ether (BDDE) with a degree of modification of approximately 1% are very poorly cohesive.

To overcome this problem, various modifications of the parameters of the process have already been tried, such as the addition of alkali halide or phosphate salts, the increase in the concentration of hyaluronic acid and/or NaOH in the cross-linking medium. Adjusting the duration and temperature of the cross-linking reaction has also been studied (WO2014/064633; WO2016/096920; WO2017/016917; Sukwha Kim and al. Facile strategy involving low-temperature chemical cross-linking to enhance the physical and biological properties of hyaluronic acid hydrogel, Carbohydrate Polymers, 2018, 202, 545-553). Nevertheless, it is always desirable to further reduce the amounts of conventional cross-linking agent used.

Moreover, hyaluronic acid gels cross-linked via Si—O—Si bonds have been prepared with a polysiloxane polymer diepoxide (Morshedi and al. Temperature-dependent formulation of a hydrogel based on Hyaluronic acid-polydimethylsiloxane for biomedical applications, Helyion, 2020, 6(3): e03494). Nevertheless, these gels have very significant mechanical properties and are therefore difficult to be injected.

Gels based on hyaluronic acid cross-linked via Si—O—Si bonds have also been prepared by functionalizing hyaluronic acid with 3-aminopropyltriethoxysilane (APTES) to form an HA-APTES then drying this HA-APTES in the presence of tetraethoxysilane (TEOS) and polydimethylsiloxane (PDMS) (Sanchez-Tellez and al., Siloxane-inorganic chemical crosslinking of hyaluronic acid-based hybrid hydrogels: Structural characterization, Carbohydrate Polymers, 2020). Nevertheless, the gels obtained are not injectable.

It has further been considered to use silicon derivatives to prepare new gels based on cross-linked biopolymers via Si—O—Si bonds, as illustrated in WO2011/089267 and WO2017/009200. Nevertheless, it should be noted that WO2011/089267 does not result in the formation of a gel with desirable mechanical properties for injection.

In addition, this type of gels using only silicon derivatives are not very stable to heat sterilization and become solutions after treatment.

Moreover, the processes described in WO2011/089267 and WO2017/009200 do not comprise such a final sterilization step. Finally, gels based on cross-linked hyaluronic acid in the presence of 1,4-butanediol diglycidyl ether (BDDE) (degree of modification of 12.8% and molar cross-linking rate of 16%) and (3-glycidyloxypropyl) trimethoxysilane (GPTMS) (degree of modification of 9% and degree of molar functionalization of 27%) have also been described with the aim of trapping active substances in an array of cross-linked hyaluronic acid to obtain their sustained release (Lee and al., One-pot synthesis of silane-modified hyaluronic acid hydrogels for effective antibacterial drugs delivery via sol-gel stabilization, Colloids and surfaces B: Biotinterfaces, 2019, 174:308-315). However, the hydrogels obtained do not have satisfactory rheological properties to be able to be injected and the amount of cross-linking agent BDDE remains very high.

There is therefore still a need to provide new polysaccharide-based hydrogels, and in particular cross-linked hyaluronic acid, which can be injected and contain smaller amounts of conventional cross-linking agent such as BDDE.

### SUMMARY OF THE INVENTION

The object of the present invention is therefore to provide hydrogels based on at least a cross-linked polysaccharide, and more particularly cross-linked hyaluronic acid, with a lower amount of conventional cross-linking agent, such as BDDE, for increased biocompatibility, with acceptable mechanical properties for the therapeutic, cosmetic and aesthetic applications covered by the present invention, in particular injectable, in particular with very good cohesiveness and good stability over time and to sterilization, in particular to heat sterilization.

In order to be able to further reduce the amount of cross-linking agent used to cross-link a polysaccharide, without impacting the mechanical properties of the composition prepared with said cross-linked polysaccharide, the inventors have discovered that it is possible to combine:
the use of a “conventional” cross-linking agent, that is to say, a cross-linking agent comprising at least two reactive functions capable of reacting with a functional group (for example, hydroxyl or carboxylic acid) of the polysaccharide (for example, hyaluronic acid), such as BDDE;
the use of molecules comprising a single reactive function capable of reacting with a functional group (for example, hydroxyl or carboxylic acid) of the polysaccharide (for example, hyaluronic acid) and at least one Si—OR function, R representing a hydrogen atom or an aliphatic hydrocarbon group, the Si—OR functions being able to condense together (once the Si—OR functions have been hydrolyzed to give Si—OH functions if necessary) to form Si—O—Si bonds resulting in additional cross-linking of said polysaccharide.

The use of such molecules having a single reactive function with respect to the polysaccharide and allowing cross-linking only via a sol-gel reaction therefore does not have the toxicity encountered with the usual cross-linking agents since these molecules are not capable of cross-linking biological molecules (proteins, DNA, etc.).

Furthermore, surprisingly, the combined presence of a silylated agent and a small amount of cross-linking agent allows to obtain a stable cross-linked gel having the desired mechanical properties, including after sterilization.

According to one embodiment, the gels prepared according to the invention have a stringy character, which is potentially advantageous in the search for muco-adhesive properties of a material.

The present invention therefore relates to a process for preparing a hydrogel, preferably injectable, comprising the following steps:
a) provision of at least a polysaccharide or a salt thereof;
b) cross-linking of the polysaccharide in the presence of 0.05 to 10 mol %, in particular 0.05 to 5 mol %, preferentially 0.1 to 2 mol % or 0.1 to 1 mol %, of at least a cross-linking agent, or a salt thereof, per 1 mole of repeat units of the polysaccharide, said cross-linking agent comprising at least two functional Z groups, which are identical or different, selected from isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue;
c) functionalization of the polysaccharide with at least a molecule of formula Chem. I:

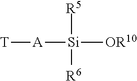

or a salt thereof in which:
T represents an isocyanate, amino, epoxide, carboxyl, N-succinimidyloxycarbonyl, N-sulfo succinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue;
A represents a chemical bond or a spacer group;
R⁵ and R⁶, which are identical or different, represent a hydrogen atom; a halogen atom; an —OR⁴ group with R⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more group(s) selected from a halogen atom, an aryl and a hydroxyl;
R¹⁰ represents a hydrogen atom, an aryl group or an aliphatic hydrocarbon group including from 1 to 6 carbon atoms;
d) sot-get reaction of at least a part of the Si-OR¹⁰ groups and optionally at least a part of the SiOR⁴ groups when they are present;
in which step b) is carried out before, or concomitantly with step c), or step b) is carried out consecutively to steps c) and d).

The present invention also relates to a hydrogel capable of being obtained by the process according to the invention.

The present invention also relates to a hydrogel, preferably injectable, comprising at least a polysaccharide cross-linked with at least one cross-linking bond LR1 and at least one cross-linking bond LR2,
the cross-linking bond LR1 comprising at least one unit Si—O—Si,
the cross-linking bond LR2, different from the cross-linking bond LR1, being obtained by cross-linking said polysaccharide with at least a cross-linking agent comprising at least two identical or different functional Z groups selected from isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue,
   said hydrogel having a degree of modification by said at least a cross-linking agent of 0.05 to 10.0%, in particular of 0.05 to 5.0%, preferentially of 0.1 to 1.0%.

The present invention also relates to a composition comprising a hydrogel according to the invention, as well as the therapeutic, cosmetic and aesthetic applications of the hydrogels according to the invention.

### Definitions

A “gel” according to the present invention is an array of polymers which is expanded throughout its volume by a fluid. This means that a gel is formed of two media, one “solid” and the other “liquid”, dispersed in each other. The medium called “solid” medium is made up of long polymer molecules interconnected by weak (for example Hydrogen) or covalent (Cross-linking) bonds and the liquid medium is made up of a solvent.

When the liquid medium serving as solvent is mainly water (for example at least 90%, in particular at least 95%, in particular at least 99% by weight), the gel is called “hydrogel”. Preferably, the liquid medium comprises, in particular consists of, a buffer solution, advantageously allowing a pH of the liquid medium comprised between 6.8 and 7.8, in particular a saline phosphate buffer.

A gel according to the present invention preferably corresponds to a product which has a phase angle δ of less than or equal to 45° at 1 Hz for a deformation of 0.1% or a pressure of 1 Pa, advantageously a phase angle δ comprised between 2° and 45°. Advantageously, some gels have a phase angle δ comprised between 20° and 45°.

Preferably, a gel according to the present invention, acceptable for the therapeutic, cosmetic and/or aesthetic applications targeted by the present invention, has a cross-over stress (or cross-over stress of the modules G′ and G″) greater than or equal to 50 Pa, preferably between 50 and 5000 Pa, and more preferably between 100 and 1000 Pa and an elastic modulus G′ greater than or equal to 20 Pa, in particular from 20 Pa to 2000 Pa, preferably from 100 Pa to 2000 Pa, more preferably from 100 Pa to 1000 Pa.

Preferably, a gel according to the present invention, acceptable for the therapeutic, cosmetic and/or aesthetic applications targeted by the present invention, has a cohesiveness of 1 N to 30 N, the cohesiveness being preferentially from 2 N to 15 N for superficial applications, and preferentially from 5 N to 20 N for deep applications. This cohesiveness is measured by mechanical compression using a rheometer. The more a gel is cohesive, that is to say the higher its cohesiveness value, the more it is capable of withstanding stresses, such as those which it may encounter after its administration to a subject.

According to the present invention a superficial application refers to the administration of a composition in the upper layers of the skin, that is to say, in or on the skin, for example by mesotherapy and for example to reduce superficial wrinkles and/or to improve the quality of the skin (such as its radiance, density or structure) and/or to rejuvenate the skin.

According to the present invention a deep application refers to the administration of a composition in the deepest layers of the skin and/or under the skin (that is to say, above the periosteum), for example to increase the volume of the soft tissues, as if to fill in deep wrinkles and/or partially atrophied regions of the face and/or body.

It should be noted that certain compositions can be versatile, that is to say, be used both for deep application and for application in the dermis between the deepest and most superficial layers of the skin, for example to reduce medium to deep wrinkles.

An “injectable” product according to the present invention corresponds to a gel which can flow and be injected manually by means of a syringe provided with a needle with a diameter comprised between 0.1 and 0.5 mm, for example with a 30 G, 27 G, 26 G, 25 G hypodermic needle). Preferably, an “injectable gel” is a gel having an average extrusion force less than or equal to 25 N, preferably 5 to 25 N, preferably 8 to 15 N, when measured with a dynamometer, at a fixed speed of approximately 12.5 mm/min, in syringes of external diameter greater than or equal to 6.3 mm, with a needle of external diameter less than or equal to 0.4 mm (27 G) and of length ½″, at room temperature.

According to the present invention, the “stringy” character of a product can be determined using a texturometer, a sensory analysis carried out by a panel, or else rheological and mechanical measurements including in particular the measurement of the phase angle (6) or tensile tests. In particular, this character can be measured as described in the example part or as described by P. Micheels and al. (Micheels and al., Comparison of two swiss-designed hyaluronic acid gels: six-month clinical follow-up, Journal of Drug in Dermatology, 2017, 16:154-161, “Resistance to stretching”).

According to the present invention, the sterilization of a hydrogel or of a composition can be carried out by heating, in particular in an autoclave (damp heat). The sterilization is advantageously carried out by increasing the temperature of the sterilization medium up to a temperature called “plateau temperature”, which is maintained for a determined duration called “plateau duration”.

According to the present invention, the sterilizing value F0 corresponds to the time required, in minutes, at 121° C., to inactivate 90% of the population of microorganisms present in the product to be sterilized.

“Monosaccharide”, also called “ose”, means, within the meaning of the present invention, an unmodified or modified monosaccharide.

An “unmodified monosaccharide” according to the invention is a compound of formula H—(CHOH)ₓ—CO—(CHOH)_{y}—H with x and y representing, independently of each other, an integer ranging from 0 to 5 on the condition that 2 s x+y s 5, the monosaccharide possibly being in a linear form represented by the aforementioned formula or possibly being in a cyclized form by reaction of the CO function (aldehyde or ketone) with the one of the OH groups to form a hemiacetal or hemiketal group. Preferably, the monosaccharide is in cyclized form. There are two types of oses: the aldoses which carry an aldehyde function (when x or y is equal to 0) and the ketoses which carry a ketone function (when neither x nor y is equal to 0). Monosaccharides are classified by number of carbons. For example, the 6-carbon monosaccharides (x+y=5) are the hexoses of formula C₆H₁₂O₆ and can be allose, altrose, glucose, mannose, gulose, idose, galactose or talose. The 5-carbon monosaccharides (x+y=4) are the pentoses of formula C₅H₁₀O₅ and can be ribose, arabinose, xylose, or lyxose. Preferably, the monosaccharide is a hexose, that is to say that x+y=5. A monosaccharide also comprises x+y asymmetric carbons and therefore 2^{(x+y-1)} pairs of enantiomers. Each pair of enantiomers is referred to by a different name and the enantiomers of the same pair are referred to as the D and L enantiomers respectively.

A “modified monosaccharide” according to the invention is an unmodified monosaccharide as defined above of which, for example:
one or more of the OH functional groups have been replaced by another functional group, for example:
   (i) an OR group with R representing a (C₁-C₆)alkyl group such as methyl or ethyl; hydroxy-(C₁-C₆)alkyl such as hydroxyethyl (—CH₂CH₂OH) or hydroxypropyl (—CH₂—CH(OH)—CH₃); carboxy-(C₁-C₆)alkyl such as carboxymethyl (—CH₂COOH); or CO—(C₁-C₆)alkyl such as acetyl; and/or
   (ii) an NR′R″ group with R′ and R″ representing, independently of one another, H, (C₁-C₆)alkyl or CO—(C₁-C₆)alkyl such as acetyl; and/or
   (iii) an OSO₃H group; and/or
the terminal CH₂OH function(s) have been replaced by a COOH or CHO group;
a —CH(OH)—CH(OH)— bond is oxidized to give two terminal —CHO (aldehyde) groups instead of this bond; and/or
a terminal CH₂OH function has been condensed with an OH functional group to form an —O—CH— chain.

A “polysaccharide” is, within the meaning of the present invention, a polymer composed of monosaccharides (preferably of the D series) joined together by glycosidic bonds.

“Repeat unit” of a polysaccharide means, within the meaning of the present invention, a structural unit consisting of one or more (generally 1 or 2) monosaccharides, the repetition of which produces the complete polysaccharide chain.

A portion or all of the monosaccharides may be in a modified form. Monosaccharides, when modified, can be in different modified forms.

Examples of polysaccharides are pectin and pectic substances, cellulose and derivatives thereof, chitosan, agarose or else glycosaminoglycans such as hyaluronic acid, heparosan and chondroitin sulfate.

A polysaccharide may be in the form of a salt, in particular in the form of a physiologically acceptable salt such as sodium salt, potassium salt, zinc salt, calcium salt, magnesium salt, silver salt and mixtures thereof, more particularly in the form of the sodium or potassium salt.

“Pectic substances”, including “pectin”, are polysaccharides composed of a skeleton of D-galacturonic acid in acid form, possibly esterified with methanol, and L-rhamnose capable of forming ramifications with other oses.

“Cellulose” is a polysaccharide composed of a linear chain of D-glucose molecules. Cellulose derivatives comprise methylcellulose, ethylcellulose, ethylmethylcellulose, hydroxypropylmethylcellulose (HPMC), hydroxyethylcellulose (HEC), hydroxypropylcellulose (HPC), carboxymethylcellulose (CMC).

“Chitosan” and “chitin” are each a polysaccharide composed of D-glucosamine repeat units bonded together in ß-(1,4) part of which is N-acetylated. Chitosan more particularly has a degree of acetylation of less than 50% while chitin more particularly has a degree of acetylation greater than 50%.

“Agarose” is a polysaccharide comprising as a repeat unit a disaccharide of D-galactose and 3,6-anhydro-L-galactopyranose.

“Glycosaminoglycans” are linear polysaccharides composed of repeat units of disaccharides, said disaccharides containing a hexosamine (glucosamine (GlcN) or galactosamine (GalN)) and another ose (glucuronic acid (GlcA), iduronic acid (IdoA) or galactose (Gal)). The hexosamine and the other ose can optionally be sulfated and/or acetylated. The glycosaminoglycan may in particular be hyaluronic acid, heparosan, or a chondroitin sulfate.

“Heparosan” is a glycosaminoglycan whose repeat unit is a disaccharide composed of glucuronic acid ((GlcA) connected by an α-(1,4) bond to an N-acetyl glucosamine (GlcNAc). Each disaccharide repeat unit is connected to the next by a ß-(1,4) bond.

“Chondroitin sulfate” is a glycosaminoglycan whose repeat unit is a disaccharide composed of glucuronic acid bonded at ß-(1,3) to N-acetyl galactosamine-6-sulfate. Each disaccharide repeat unit is bonded to the next by a ß-(1,4) bond.

“Hyaluronic acid” is a glycosaminoglycan whose repeat unit is a disaccharide composed of D-glucuronic acid and N-acetyl-D-glucosamine, bonded together by alternating ß-(1,4) and ß-(1,3) glycosidic bonds. When the hyaluronic acid is in the form of a salt, it is also referred to as “hyaluronate” or “hyaluronan”. In the context of the present invention, the hyaluronic acid may have a weight-average molecular mass comprised between 0.05 and 10 MDa, preferably between 0.5 and 5 MDa, for example between 0.5 and 4 MDa or between 0.5 and 2 MDa. The hyaluronic acid can be in the form of a salt, in particular in the form of a physiologically acceptable salt such as sodium salt, potassium salt, zinc salt, calcium salt, magnesium salt, silver salt, calcium salt and mixtures thereof. More specifically, the hyaluronic acid is in its acid or sodium salt (NaHA) form.

In the context of the present invention, a cross-linking reaction of a polysaccharide is a reaction allowing the formation of covalent bonds, cross-linking bonds, in or between the polysaccharide chains.

In the context of the present invention, a “cross-linking agent” or “agent for cross-linking” is a compound comprising at least two functional groups capable of bonding covalently with functional groups present on the polysaccharide, such as OH, CHO, NH₂ or COOH groups, and thus induce cross-linking between the polysaccharide chains. In particular, a cross-linking agent according to the invention will comprise at least two, preferably from 2 to 8, in particular 2, functional groups preferably selected from isocyanate (—N═C═O), amino (—NH₂), epoxide, carboxyl (—COOH), N-succinimidyloxycarbonyl, N-sulfosuccinimidyloxycarbonyl, halogenocarbonyl, isothiocyanate (—N═C═S), vinyl (—CH═CH₂), formyl (—CH═O), hydroxyl (—OH), sulfhydryl (—SH), hydrazino (—NH—NH₂), acylhydrazino (—CO—NH—NH₂), aminoxy (—O—NH₂), carbodiimide groups, and an acid anhydride residue.

An “epoxide” group is an ethylene oxide residue bonded to the rest of the molecule by one of its carbon atoms.

An “N-succinimidyloxycarbonyl” group is a group of formula Chem. GR1 below:

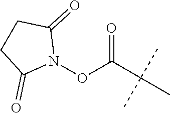

An “N-sulfosuccinimidyloxycarbonyl” group is a group of formula Chem. GR2 below:

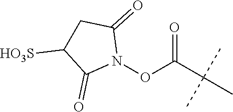

A “halogenocarbonyl” group is a group of formula —CO-Hal with Hal representing a halogen, such as Cl or Br.

A “carbodiimide” group is a group comprising a unit —N═C═N—, and more particularly a group of formula —N═C═NR^{a} with R^{a} representing an aliphatic hydrocarbon group including from 1 to 20 carbon atoms, preferably a (C1-C6)alkyl group, one or more carbon atoms of which are optionally replaced by a heteroatom selected from 0, S and N, in particular N.

An “acid anhydride residue” is a group comprising a unit —C(O)—O—C(O)—, and more particularly a monovalent cyclic group comprising the unit —C(O)—O—C(O)—, such as a saturated monovalent hydrocarbon monocyclic group comprising 5 to 10, in particular 5 or 6, carbon atoms of which three successive carbon atoms are replaced by C(O)—O—C(O) and optionally of which one or more, in particular one, additional carbon atoms, preferably not consecutive to the three carbon atoms substituted by CO—O—CO, are each replaced by a heteroatom such as N, O or S, in particular N. The acid anhydride residue can meet in particular the following formula Chem. GR3:

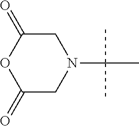

The acid anhydride residue can also be selected from a maleic anhydride residue or a succinic anhydride residue.

In particular, the isocyanate group can react with an OH or NH₂ group of the polysaccharide to form a carbamate or urea function, the amino group can react with a COOH group of the polysaccharide to form an amide function, the epoxy group can react with an OH or COOH group of the polysaccharide to form an ether or ester function, the carboxyl group can react with an OH or NH₂ group of the polysaccharide to form an ester or amide function, the N-succinimidyloxycarbonyl and N-sulfosuccinimidyloxycarbonyl groups can react with an OH or NH₂ group of the polysaccharide to form an ester or amide function, the halogenocarbonyl group can react with an OH or NH₂ group of the polysaccharide to form an ester or amide function, the isothiocyanate group can react with an OH or NH₂ group of the polysaccharide to form a thiocarbamate or thiourea function, the vinyl group can react with an OH group of the polysaccharide to form an ether function, the formyl group can react with an OH or NH₂ group to form a hemiacetal or hemiaminal function, a hydroxyl group can react with a COOH group of the polysaccharide to form an ester function, the sulfhydryl group can react with a COOH group of the polysaccharide to form a thioester function, the hydrazino group (—NH—NH₂) can react with a CHO group to form a hydrazone function, the acylhydrazino group can react with a CHO group of the polysaccharide to form a carbonyl hydrazone function ═NNHC(O)—, the aminoxy group can react with a CHO group of the polysaccharide to form an oxime ═NO— function, the carbodiimide group can react with a COOH group of the polysaccharide to give a CO—NR^{a}—CO—NH function, and an acid anhydride residue can react with an OH or NH₂ group of the polysaccharide to form an ester or amide function.

“Spacer group” according to the present invention means a fragment comprising at least one atom. Preferably, the spacer group contains at least one carbon atom. The spacer group aims at binding together two chemical groups within the same molecule, here in particular the molecule of formula Chem. I. Advantageously, the spacer group will also allow to avoid steric hindrance between the silyl group and the T group of the molecule of formula Chem. I, while ensuring a stable bond between these two groups. The term “halogen”, as used in the description of the present invention, refers to the atoms of fluorine, chlorine, bromine and iodine. Advantageously, it will be fluorine, bromine or chlorine.

In the present invention, the terms “aliphatic hydrocarbon chain” or “aliphatic hydrocarbon group” designate linear, branched and/or cyclic, saturated or unsaturated but non-aromatic hydrocarbon groups, advantageously comprising 1 to 50, in particular 1 to 20, for example 1 to 12 or 1 to 6 carbon atoms. Said groups will in particular be alkyl groups.

In the present invention, the term “branched aliphatic hydrocarbon chain” is understood to mean a main aliphatic hydrocarbon chain also comprising at least one secondary aliphatic hydrocarbon chain.

In the present invention, the term “starred aliphatic hydrocarbon chain” is understood to mean a branched aliphatic hydrocarbon chain comprising several secondary aliphatic hydrocarbon chains all starting from a single branching point.

In the present invention, the term “C1-Cx alkyl”, “(C1-Cx)alkyl” or else “alkyl including from 1 to x carbon atoms”, is understood to mean a monovalent saturated hydrocarbon group, which is linear or branched, including from 1 to x carbon atoms, with x an integer, such as for example a methyl, ethyl, isopropyl, tert-butyl, n-pentyl, cyclopropyl, cyclohexyl group, etc.

“(C1-Cx)alkylene” group means a divalent saturated hydrocarbon group, linear or branched, including from 1 to x carbon atoms, with x an integer, such as for example a methane-1,1-diyl, ethane-1,1-diyl, ethane-1,2-diyl, propane-1,3-diyl, butane-1,4-diyl, butane-1,3-diyl, butane-1,2-diyl, pentane-1,5-diyl, hexane-1,6-diyl, hexane-1,5-diyl, heptane-1,7-diyl, octane-1,8-diyl, nonane-1,9-diyl, decane-1,10-diyl group, etc. This is in particular a methane-1,1-diyl or propane-1,3-diyl group.

“Hydroxy-(C1-Cx)alkyl” group means a (C1-Cx)alkyl group as defined above substituted by a hydroxyl group (OH) such as for example a hydroxyethyl (—CH₂CH₂OH) or hydroxypropyl (for example —CH₂—CH(OH)—CH₃).

“Carboxy-(C1-Cx)alkyl” group means a (C1-Cx)alkyl group as defined above substituted by a carboxyl (COOH) group such as for example a carboxymethyl (—CH₂COOH).

“Aryl” group means a monovalent aromatic hydrocarbon group, preferably including from 6 to 10 carbon atoms, comprising one or more cycles, such as for example a phenyl or naphthyl group.

“Arylene” group means a divalent aromatic hydrocarbon group, preferably including from 6 to 10 carbon atoms, comprising one or more cycles, such as a phenylene group.

The term “aryl-(C1-Cx)alkyl”, as used in the description of the present invention, denotes an aryl group as defined above, bonded to the rest of the molecule via a chain (C1-Cx)alkyl as defined above with x an integer. By way of example, mention may be made of the benzyl or else phenylethyl group.

A “polyvalent group” according to the present invention is a group which can form several covalent bonds with other groups of the same compound. The bonds to the other groups can be formed from the same atom of the polyvalent group or from different atoms of the polyvalent group, and preferably from different atoms of the polyvalent group. In particular, the polyvalent group is a divalent group and can therefore form two covalent bonds with two other groups of the same compound or of two different compounds. The number of covalent bonds that can be formed refers to the “valence” of the polyvalent group.

The expression “degree of molar modification” designates the molar ratio of the amount of modifying agent (for example cross-linking agent or molecule of formula Chem. I as functionalizing agent) relative to the amount of repeat unit of the polysaccharide used in the modification medium.

More specifically, the expression “molar cross-linking rate” (%) designates the molar ratio of the amount of cross-linking agent relative to the amount of repeat unit of the polysaccharide used in the cross-linking medium, that is to say the medium of step b), expressed for 100 moles of repeat units of polysaccharide in the cross-linking medium; the expression “molar functionalization rate” (%) designates the molar ratio of the amount of functionalizing agent (for example, molecule of formula Chem. I) relative to the amount of repeat unit of the polysaccharide used in the functionalization medium, that is to say the medium of step c), expressed for 100 moles of repeat units of the polysaccharide in the functionalization medium.

The expression “mass modification rate” designates the mass ratio of the mass of modifying agent (for example, cross-linking agent or molecule of formula Chem. I as functionalizing agent) relative to the sum of the mass of modifying agent and the mass of polysaccharide in the modification medium.

More specifically, the expression “mass cross-linking rate” (%) designates the mass ratio of the mass of cross-linking agent relative to the sum of the mass of cross-linking agent and of the mass of polysaccharide in the cross-linking medium, that is to say in the medium of step b), expressed as percentages; the expression “mass functionalization rate” (%) designates the mass ratio of the mass of functionalizing agent (for example, molecule of formula Chem. I) relative to the sum of the mass of functionalizing agent and the mass of polysaccharide in the functionalization medium, that is to say in the medium of step c), expressed in percentages.

The “degree of modification” (abbreviated MOD, %) corresponds to the molar amount of modifying agent, such as cross-linking agent or functionalizing agent (for example, molecule of formula Chem. I), bound to the polysaccharide, by one or more of its ends, expressed for 100 moles of repeat units of the polysaccharide. It can be determined by processes known to the person skilled in the art such as Nuclear Magnetic Resonance (NMR) spectroscopy. For example, a MOD of 1% means that there is one molecule of modifying agent for 100 moles of repeat units of the polysaccharides.

In the context of the present invention, the “sol-gel reaction” consists in forming Si—O—Si bonds from Si—OR groups with R representing a hydrogen atom, an aryl group or an aliphatic hydrocarbon group including from 1 to 6 carbon atoms. This reaction proceeds as follows:
(i) if R is not a hydrogen atom, a hydrolysis step of at least some of the Si—OR groups to give Si—OH groups; then
(ii) a step of condensation of the Si—OH groups in pairs or of an Si—OH group with an Si—OR group to form Si—O—Si bonds.

In the present invention, the Si—OR groups are carried in particular by polysaccharides functionalized with at least a molecule of formula Chem. I during step c).

“Physiologically acceptable” means anything that is generally safe, non-toxic and neither biologically nor otherwise undesirable and which is acceptable for cosmetic or aesthetic (that is to say non-therapeutic) or therapeutic human or veterinary use, in particular for use by injection into the human or animal body or for topical application to the skin.

The “salts” used in the context of the present invention are preferably physiologically acceptable.

Physiologically acceptable salts comprise in particular:
(1) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as formic acid, acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, and
(2) pharmaceutically acceptable base addition salts formed when an acidic proton present in the parent compound is either replaced by a metal ion, for example an alkali metal ion (for example, Na, K), an alkaline-earth metal ion (for example, Ca, Mg), a zinc ion, a silver ion or an aluminum ion; is coordinated with a pharmaceutically acceptable organic base such as diethanolamine, ethanolamine, N-methylglucamine, triethanolamine, tromethamine and the like; or with a pharmaceutically acceptable inorganic base such as aluminum hydroxide, calcium hydroxide, potassium hydroxide, sodium carbonate, sodium hydroxide and the like.

### DETAILED DESCRIPTION

Process

The present invention relates to a process for preparing a hydrogel described above, comprising steps a), b), c) and d). This process is advantageously carried out in an aqueous medium.

In particular, the hydrogel is an injectable hydrogel.

Step a)

Step a) of the process according to the invention comprises providing at least a polysaccharide or a salt thereof.

Preferably, the polysaccharide is selected from pectin and pectic substances; chitosan; cellulose and derivatives thereof; agarose; glycosaminoglycans such as hyaluronic acid, heparosan and chondroitin sulfate; and mixtures thereof.

In particular, the cellulose derivatives are selected from methylcellulose, ethylcellulose, ethylmethylcellulose, hydroxypropylcellulose (HPC), hydroxypropylmethylcellulose (HPMC), hydroxyethylcellulose (HEC), and carboxymethylcellulose (CMC).

Advantageously, the polysaccharide is a glycosaminoglycan, preferably hyaluronic acid or a salt thereof, more preferably hyaluronic acid or one of its physiologically acceptable salts such as sodium salt, potassium salt, zinc salt, silver salt and mixtures thereof, even more preferably hyaluronic acid or its sodium salt.

Preferably, the polysaccharide has a weight-average molecular mass Mw comprised between 0.05 and 10 MDa, preferentially between 0.5 and 5 MDa, for example between 0.5 and 4 MDa or between 0.5 and 2 MDa.

In particular, in step a) the polysaccharide is supplied in hydrated form, totally or partially, or in dry form, such as in powder or fiber form. More particularly, in step a), the polysaccharide is provided in dry form such as in powder or fiber form.

When the polysaccharide is provided in hydrated form, it is in the form of a non-cross-linked gel or a solution.

In particular, when the polysaccharide is in hydrated form, it is an aqueous non-cross-linked gel or an aqueous solution. More particularly, the polysaccharide is mixed with water, optionally added with a phosphate buffer or with a supplemented phosphate buffer, that is to say possibly comprising additional components as defined in step f), or an alkaline medium adapted for step b).

Step b)

Step b) of the process according to the invention comprises the cross-linking of the polysaccharide provided in step a) in the presence of 0.05 to 10 mol % of at least a cross-linking agent per 1 mole of repeat unit of the polysaccharide, said cross-linking agent comprising at least two functional Z groups as described below.

When the polysaccharide is a glycosaminoglycan such as hyaluronic acid, the repeat unit is therefore a disaccharide unit.

The functional Z groups, which are identical or different, are selected from isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue.

Advantageously, the functional Z groups are identical.

Preferably, the functional Z groups are identical and represent an epoxide or vinyl group, more preferably epoxide.

According to another advantageous embodiment, the functional Z groups are identical and selected from amino, vinyl, formyl and carbodiimide groups, preferably are amino groups.

In particular, the cross-linking agent is selected from hexamethylene diisocyanate, diphenylmethylene 4,4′-diisocyanate, 4-arm PEG20K-isocyanate, spermine (or 1,12-diamino-5,9-diazadodecane), spermidine (or 1,8-diamino-5-azaoctane), cadaverine (or 1,5-diaminopentane), putrescine (or 1,4-diaminobutane), poly(ethylene glycol) diamine, ethylenediamine, 1,4-butanediol diglycidyl ether (BDDE), 1,2,7,8-diepoxy-octane, poly(ethylene glycol) diglycidyl ether (PEGDGE), 1,2-bis(2,3-epoxypropoxy) ethane (EGDGE), 1,3-bis(3-glycidyloxypropyl) tetramethyldisiloxane, poly(dimethylsiloxane) terminated at each end with a diglycidyl ether (CAS number: 130167-23-6), poly(ethylene glycol) diacid, disuccinimidyl suberate, bis(sulfosuccinimidyl)suberate, sebacoyl chloride, 1,4-butane diisothiocyanate, divinylsulfone (DVS), glutaraldehyde, polyethylene glycol, 1,5-pentanedithiol, adipic acid dihydrazid, bis-aminooxy-poly(ethylene glycol), diethylenetriaminepentaacetic acid dianhydride, and mixtures thereof.

When the functional Z groups are epoxide groups, the cross-linking agent is preferably selected from 1,4-butanediol diglycidyl ether (BDDE), 1,2,7,8-diepoxy-octane, poly (ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end with a diglycidyl ether (CAS number: 130167-23-6), hydroxyapatite beads modified to carry epoxy groups and mixtures thereof.

More preferably, the cross-linking agent is selected from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and mixtures thereof.

When the functional Z groups are amino groups, the cross-linking agent is preferably a polyamine selected from spermine (or 1,12-diamino-5,9-diazadodecane), spermidine (or 1,8-diamino-5-azaoctane), cadaverine (or 1,5-diaminopentane), putrescine (or 1,4-diaminobutane), salts thereof or a mixture thereof, more preferably the cross-linking agent is a polyamine selected from spermine, spermidine, salts thereof and mixtures thereof.

When the functional Z groups are amino groups, the cross-linking reaction with the polysaccharide is advantageously carried out in the presence of at least an activator, and where appropriate combined with at least a coupling auxiliary.

In this regard, the activator can be selected from water-soluble carbodiimides such as 1-ethyl-3-(3-dimethylaminopropyl)carbodiimide (EDC), 1-ethyl-3-[3-(trimethylamino) propyl]carbodiimide hydrochloride (ETC), 1-cyclohexyl-3-(2-morphilinoethyl)carbodiimide (CMC), salts thereof and mixtures thereof, preferably is represented by EDC.

As regards the coupling auxiliary, when present, it can be selected from N-hydroxy succinimide (NHS), N-hydroxybenzotriazole (HOBt), 3,4-dihydro-3-hydroxy-4-oxo-1,2,3-benzotriazole (HOOBt), 1-hydroxy-7-7azabenzotriazole (HAt) and N-hydroxysylfosuccinimide (sulfo NHS), and mixtures thereof, preferably is represented by HOBt.

The cross-linking agent can be selected from hydroxyapatite beads modified to carry epoxy groups, a compound of formula Chem. II as described below, and mixtures thereof.

Preferably, the cross-linking agent is a compound of formula Chem. II:

Y—(Z)ₙ

in which the Z groups, which are identical or different, are as defined above, n is an integer greater than or equal to 2, in particular ranging from 2 to 8, preferably equal to 2,
Y is a polyvalent hydrocarbon group, in particular aliphatic, having a valence of n and including from 1 to 150 carbon atoms:
in which one or more (for example 1 to 150, or else 1 to 50 or else 1 to 15 or else 1 or 2) units CH₂ are optionally replaced by one or more divalent units selected from arylenes; —O—; —S—; —S(O)—; —C(═O)—; —SO₂—; —N(R¹)—; and —[SiR²R³O]ₘ—SiR²R³— with:
R¹ representing a hydrogen atom, an aliphatic hydrocarbon group including from 1 to 6 carbon atoms, or an aryl-(C1-C6)alkyl;
m an integer comprised between 1 and 20 and
the R² and the R³, which are identical or different, representing a hydrogen atom; a halogen atom; an —OR¹¹ group with R¹¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl or a hydroxyl,
said polyvalent group being unsubstituted or substituted by one or more monovalent groups selected from a halogen atom, a hydroxyl, and an aryl- (C1-C6)alkyl, preferably unsubstituted.

In particular n is an integer ranging from 2 to 8, preferably n represents 2, 3 or 4, even more preferably n is equal to 2.

Advantageously, R¹ represents a hydrogen atom or a (C1-C6)alkyl group.

In particular, R² and R³, which are identical or different, represent an aliphatic hydrocarbon group including from 1 to 6 carbon atoms, more particularly a (C1-C6)alkyl group.

Preferably, in the definition of Y, the hydrocarbon polyvalent group may be an aliphatic or aromatic hydrocarbon polyvalent group, preferably aliphatic and in particular saturated, having a valence of n and including from 1 to 150 carbon atoms, preferentially from 1 to 50 carbon atoms, more preferably from 1 to 20 carbon atoms, even more preferably from 2 to 20 carbon atoms.

In particular, in the definition of Y, the hydrocarbon polyvalent group is a linear, saturated, aliphatic hydrocarbon polyvalent group.

Preferably, Y is a hydrocarbon polyvalent group as described above in which one or more units CH₂ are optionally replaced by one or more divalent units selected from —O—, —SO₂, —[SiR²R₃O]ₘ—SiR²R³— and —NH—, with R², R³ and m as described above.

In particular, Y is a hydrocarbon polyvalent group as described above, preferably aliphatic and saturated, and in particular linear, branched, or starred, and optionally in which:
at least two units CH₂ are replaced by —O—, particularly between 1 and 50 units CH₂, more particularly between 1 and 15 units CH₂, or
at least one, preferably one or two, units CH₂ is replaced by a unit —NH—, or
at least one, preferably one, unit CH₂ is replaced by a unit —SO₂—, or
at least two, preferably two, units CH₂ are replaced by —O— and at least one, preferably one, unit CH₂ is replaced by a unit —[SiR²R³O]ₘ—SiR²R³— with R², R³ and m as described above.

More particularly, when one or more units CH₂ are replaced by —O—, the replaced unit(s) are such that Y comprises one or more —CH₂—CH₂—O— units. In particular, Y comprises from 1 to 50 units —CH₂—CH₂—O—, advantageously from 2 to 25 units —CH₂—CH₂—O—, more advantageously from 2 to 15 units —CH₂—CH₂—O—. Y can comprise only units —CH₂—CH₂—O—. More preferably, Y is an alkyl group comprising 1 to 150, in particular 1 to 50, in particular 1 to 20, for example 1 to 12, in particular 1 to 6 carbon atoms, preferably linear, in which optionally one or more units CH₂ are replaced by one or more divalent units selected from —O— and —NH—, more particularly between 1 and 50, in particular between 1 and 15, for example 1 or 2, divalent units selected from —O— and —NH—.

According to a first embodiment, the R² and the R³, which are identical or different, represent an —OR¹¹ group with R¹¹ as described above. In particular, R¹¹ represents an aliphatic hydrocarbon group including from 1 to 6 carbon atoms, more particularly a (C1-C6)alkyl group.

According to a second embodiment, the R² and the R³, which are identical or different, represent an aliphatic hydrocarbon group including from 1 to 6 carbon atoms optionally substituted (preferably unsubstituted) by one or more groups selected from a halogen atom, an aryl or a hydroxyl, more preferably an unsubstituted (C1-C6)alkyl group such as methyl or ethyl.

Advantageously, the cross-linking agent is a compound having the following formula Chem. IIa:

Z¹—Y¹—Z²

in which the Z¹ and Z² groups, which are identical or different, are selected from isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue,

and Y¹ represents a divalent hydrocarbon chain, in particular aliphatic, including from 1 to 50 carbon atoms:
in which one or more (for example 1 to 15 or else 1 or 2) units CH₂ are optionally replaced by one or more divalent units selected from arylenes, —O—, —S—, —S(O)—, —C(═O)—, —SO₂—, —N(R¹)—, and —[SiR²R³O]ₘ—SiR²R³— with
R¹ representing a hydrogen atom, an aliphatic hydrocarbon group including from 1 to 6 carbon atoms, or an aryl-(C1-C6)alkyl,
m an integer comprised between 2 and 20, and
the R² and the R³, which are identical or different, representing a hydrogen atom; a halogen atom; a —OR¹¹ group with R¹¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl or a hydroxyl,
said chain being unsubstituted or substituted by one or more monovalent groups selected from a halogen atom, a hydroxyl, an aryl-(C1-C6)alkyl.

The Z¹ and Z² groups have the same definition as the Z group defined above.

Y¹ has the same definition as Y defined above with a valence n being equal to 2.

In particular, Y¹ can only comprise units —CH₂—CH₂—O—, as defined previously.

Preferably, the cross-linking agent of formula Chem. II or Chem. IIa does not comprise units —[SiR²R³O]ₘ—SiR²R³—.

The cross-linking of the polysaccharide provided in step a) takes place in the presence of 0.05 to 10 mol %, in particular from 0.05 to 7 mol %, more advantageously from 0.05 to 5 mol %, even more advantageously from 0.1 to 2 mol % and in particular from 0.1 to 1 mol %, of at least a cross-linking agent per 1 mole of repeat unit of the polysaccharide.

The mass concentration of polysaccharide in the cross-linking medium is advantageously comprised between 50 and 300 mg/g of cross-linking medium, preferably between 100 and 200 mg/g.

In particular, the cross-linking takes place in an aqueous reaction medium. However, if necessary, an organic solvent such as an alcohol, in particular ethanol, or DMSO can be used to solubilize the cross-linking agent, for example when it comes to the poly(dimethylsiloxane) terminated at each end with a diglycidyl ether (CAS number: 130167-23-6) before addition to the aqueous reaction medium.

Advantageously, and in particular when the Z groups, such as Z¹ or Z², represent an epoxy group or a vinyl group, the cross-linking takes place at a pH greater than or equal to 10, more advantageously greater than or equal to 12.

For this purpose, the reaction medium preferably comprises a Bronsted base, more preferably a hydroxide salt, such as sodium or potassium hydroxide.

Preferably, the cross-linking takes place between 4° C. and 60° C., more preferably between 10° C. and 50° C.

In particular, the cross-linking takes place between 1 hour and 2 weeks, more particularly between 3 hours and 1 week.

In the presence of several cross-linking agents, the cross-linking agents can be added simultaneously or separately over time. Step b) can thus comprise repeated cross-linking steps. The total cross-linking rate in cross-linking agents varies from 0.05 to 10% molar as defined previously.

This step allows to cross-link the polysaccharide chains with each other. The functional groups of the cross-linking agent react with functional groups present on the polysaccharides so as to bond the polysaccharide chains with each other and to cross-link them by forming intermolecular bonds. The cross-linking agent can also react with functional groups present on the same polysaccharide molecule so as to form intramolecular bonds. In particular, the functional groups of the cross-linking agent react with the —OH or —COOH, or else —CHO groups, present on the polysaccharides such as hyaluronic acid. Cross-linked polysaccharides comprising at least one cross-linking bond between two polysaccharide chains are thus obtained, said cross-linking bond being the residue of the cross-linking agent from step b).

In particular, following step b), the cross-linked polysaccharides comprise at least one cross-linking bond between two polysaccharide chains, said cross-linking bond comprising more particularly the polyvalent Y group as described above, preferably the divalent Y¹ group as described above.

Some functional Z (such as Z¹ and Z²) groups of the cross-linking agent may however not react with a polysaccharide chain.

In particular, when the cross-linking agent includes two functional Z¹ and Z² groups, one of the functional Z¹ groups can react with a polysaccharide while the other functional Z² group does not react with any polysaccharide. A dangling bond is then formed.

Step c)

Step c) of the process according to the invention comprises the functionalization of the polysaccharide with at least a molecule having the following formula Chem. I:

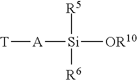

or a salt thereof,
in which:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, acylhydrazino, aminoxy, carbodiimide group, or an acid anhydride residue;
A represents a chemical bond or a spacer group;
R⁵ and R⁶, which are identical or different, represent a hydrogen atom; a halogen atom; an —OR⁴ group with R⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl and a hydroxyl;
R¹⁰ represents a hydrogen atom, an aryl group or an aliphatic hydrocarbon group including from 1 to 6 carbon atoms.

Advantageously, T represents an isocyanate, sulfhydryl, amino, epoxide, vinyl, formyl or carbodiimide group, more advantageously, T represents an epoxide or amino group, even more advantageously T represents an epoxide group.

In particular, the T group of the molecule of formula Chem. I and the Z or Z¹ and Z² groups of the molecule of formula Chem. II/Chem. IIa are identical.

Preferably, A represents a spacer group, more preferably a divalent aliphatic hydrocarbon chain, in particular linear or branched and saturated, including from 1 to 12 carbon atoms:
in which are optionally interposed, between two carbon atoms of said chain, one or more (in particular 1, 2, 3 or 4) divalent units selected from arylenes, —O—, —S—, —S(O)—, —C(═O)—, —SO₂— and —N(R⁹)— with R⁹ representing a hydrogen atom, an aliphatic hydrocarbon group including from 1 to 6 carbon atoms, or an aryl-(C1-C6)alkyl,
said chain being unsubstituted or substituted by one or more monovalent groups selected from a halogen atom, a hydroxyl, an aryl-(C1-C6)alkyl.

Advantageously, A is a divalent aliphatic hydrocarbon chain, in particular linear or branched and saturated, in which are optionally interposed, between two carbon atoms of said chain, one or more divalent units —O—, more advantageously from 1 to 4 divalent units —O—, even more preferably one divalent unit O.

Preferably, A is a (C1-C12)alkylene chain in which are optionally interposed, between two carbon atoms of said chain, one or more divalent units —O—, more preferably from 1 to 4 divalent units —O—, even more preferably one divalent unit —O—.

In particular, A represents a divalent chain —(C1-C6)alkylene-O—(C1-C6)alkylene-, in particular —(C1-C4)alkylene-O—(C1-C4)alkylene-, more particularly a divalent chain —CH₂—O—(CH₂)₃—, the CH₂ group being bonded to T and the (CH₂)₃ group being bonded to Si in the molecule of formula Chem. I.

Advantageously, R⁵ and R⁶, which are identical or different, represent an —OR⁴ group with R⁴ representing a hydrogen atom, an aryl group or an aliphatic hydrocarbon group including from 1 to 6 carbon atoms; or an aliphatic hydrocarbon group including from 1 to 6 carbon atoms optionally substituted by one or more groups selected from a halogen atom, an aryl and a hydroxyl.

In particular, R⁵ and R⁶, which are identical or different, represent an —OR⁴ group with R⁴ representing a (C1-C6)alkyl group; or a (C1-C6)alkyl group.

Advantageously, R⁵ and R⁶, which are identical or different, represent an —OR⁴ group with R⁴ representing a hydrogen atom, an aryl group or an aliphatic hydrocarbon group including from 1 to 6 carbon atoms, preferably with R⁴ representing an aliphatic hydrocarbon group including from 1 to 6 carbon atoms, such as a (C1-C6)alkyl group.

Advantageously, R¹⁰ represents a hydrogen atom or an aliphatic hydrocarbon group including from 1 to 6 carbon atoms such as a (C1-C6)alkyl group, more advantageously R¹⁰ represents an aliphatic hydrocarbon group including from 1 to 6 carbon atoms such as a (C1-C6)alkyl group.

Preferably, the molecule of formula Chem. I is such that:
T is as defined above and advantageously represents an amino or epoxide group, preferably an epoxide group;
A is a divalent chain —(C1-C6)alkylene-O—((C1-C6)alkylene-, in particular —(C1-C4)alkylene-O—(C1-C4)alkylene-, such as —CH₂—O—(CH₂)₃—, the CH₂ group preferably being bonded to T and the (CH₂)₃ group being bonded to Si in the molecule of formula Chem. I;
R⁵ and R⁶, which are identical or different, are each an —OR⁴ group with R⁴ representing a (C1-C6)alkyl group, preferably a methyl or an ethyl; or a (C1-C6)alkyl group, preferably a methyl or an ethyl; and
R¹⁰ is a (C1-C6)alkyl group, preferably methyl or ethyl;
the R⁵, R⁶ and OR¹⁰ groups possibly being identical.

In particular, the molecule of formula Chem. I is selected from (3-glycidyloxypropyl)trimethoxysilane (GPTMS), 3-Glycidoxypropyldimethoxymethylsilane, 3-Glycidoxypropyldimethylethoxysilane, (3-glycidyloxypropyl)ethoxydimethoxysilane, (3-glycidyloxypropyl)triethoxysilane, diethoxy(3-glycidyloxypropyl)methylsilane, and mixtures thereof; preferably from (3-glycidyloxypropyl)trimethoxysilane (GPTMS), (3-glycidyloxypropyl)ethoxydimethoxysilane, (3-glycidyloxypropyl)triethoxysilane, diethoxy(3-glycidyloxypropyl)methylsilane, and mixtures thereof.

Preferably, in step c), the polysaccharide is functionalized in the presence of 5 to 50 mol %, preferentially of 10 to 45 mol %, in particular of 20 to 45 mol %, for example of 30 mol % to 45 mol %, or else in the presence of 5 to 25 mol %, in particular of 10 to 25 mol %, for example of 15 mol % to 25 mol %, of molecule of formula Chem. I or a salt thereof per 1 mole repeat unit of the polysaccharide.

The mass concentration of polysaccharide in the functionalization medium is advantageously comprised between 50 and 300 mg/g of functionalization medium, preferably between 100 and 200 mg/g.

In general, the amount of functionalizing agent increases when the amount of cross-linking agent is reduced, and vice versa.

Advantageously, the cross-linking of the polysaccharide takes place in step b) in the presence of 0.05 to 2 mol % or 0.05 to 1 mol % of at least a cross-linking agent per 1 mole of repeat unit of the polysaccharide, and the functionalization of the polysaccharide takes place in step c) in the presence of 10 mol % to 50 mol %, in particular from 10 mol % to 45 mol %, preferably from 30 mol % to 45 mol %, or else in the presence of 5 mol % to 50 mol %, in particular from 10 mol % to 25 mol %, preferably from 15 mol % to 25 mol %, of molecule of formula Chem. I or a salt thereof per 1 mole repeat unit of the polysaccharide.

Advantageously, the cross-linking of the polysaccharide takes place in step b) in the presence of 2 mol % to 10 mol % or 1 to 10 mol % of at least a cross-linking agent per 1 mole of repeat unit of the polysaccharide, and the functionalization of the polysaccharide takes place in step c) in the presence of 5 mol % to 50 mol %, in particular from 10 mol % to 45 mol %, preferably from 5 mol % to 25 mol % or 20 mol % or 45 mol % of molecule of formula Chem. I or a salt thereof per 1 mole repeat unit of the polysaccharide.

In particular, the functionalization takes place in an aqueous reaction medium.

Advantageously, and in particular when T is an epoxide, the functionalization takes place at a pH greater than or equal to 10, more advantageously greater than or equal to 12.

For this purpose, the reaction medium preferably comprises a Bronsted base, more preferably a hydroxide, even more preferably a sodium or potassium hydroxide.

Preferably, the functionalization takes place between 4° C. and 60° C., more preferably between 10° C. and 50° C.

In particular, the functionalization takes place between 1 hour and 2 weeks, more particularly between 3 hours and 1 week.

Step c) allows to functionalize the polysaccharide chains. The functional group T of the molecule of formula Chem. I thus reacts with a functional group present on the polysaccharides so as to functionalize the polysaccharide chains. In particular, the T functional group of the molecule Chem. I thus reacts with an —OH or —COOH group, or else a CHO function, present on polysaccharides such as hyaluronic acid.

Functionalized polysaccharides comprising dangling bonds on a polysaccharide chain are thus obtained, said dangling bonds comprising an -A-Si(R⁵)(R⁶)OR¹⁰ group, the -A-Si(R⁵)(R⁶)OR¹⁰ group from the molecule of formula Chem. I of step c) which can provide biological properties to the hydrogel.

Advantageously, steps b) and c) are concomitant.

Step d)

Step d) of the process according to the invention comprises a sol-gel reaction of at least part of the Si—OR¹⁰ groups and optionally of at least part of the SiOR⁴ groups when they are present.

This step also allows to cross-link the polysaccharide chains with one another when they are functionalized with molecules of formula Chem. I. Indeed, during this step, at least part of the Si—OR¹⁰ groups and optionally at least part of the SiOR⁴ groups will react in pairs, possibly after hydrolysis of these groups, to form Si—O—Si bonds. This implies that two molecules with the formula Chem. I grafted onto polysaccharide chains will react together via their terminal Si—OR¹⁰ (or even SiOR⁴ if necessary) groups and bind covalently via the formation of Si—O—Si bonds, thus allowing to bind the polysaccharide chains together and further cross-link them.

Cross-linked polysaccharides are thus obtained comprising cross-linking bonds between two polysaccharide chains, said cross-linking bonds comprising a divalent —Si—O—Si— group.

Therefore, step d) cannot take place before step c). It is in particular at least partly concomitant with step c).

As defined previously, the sol-gel reaction comprises one or two sub-steps, namely a hydrolysis sub-step d1) carried out only when R¹⁰ (or R⁴) is not a hydrogen atom and a condensation sub-step d2).

Advantageously, the hydrolysis step d1) takes place at a pH greater than 9, preferably greater than 10, more preferably greater than 12.

For this purpose, the reaction medium preferably comprises a Bronsted base, more preferably a hydroxide, even more preferably a sodium or potassium hydroxide.

Preferably, the hydrolysis takes place between 4° C. and 60° C., more preferably between 10° C. and 50° C.

In particular, the hydrolysis takes place between 1 hour and 2 weeks, more particularly between 3 hours and 1 week.

Step c) and step d1), when the latter takes place, are therefore preferably concomitant since they can be carried out under the same reaction conditions.

The condensation step d2) can take place at a pH greater than 9 but with slow kinetics.

This is why it is preferable to use other reaction conditions to promote this condensation, such as in particular lowering the pH to a value for example ranging from 6.8 to 7.8 or partially or completely drying (in particular dehydrating when the reaction medium is aqueous) the reaction medium (for example by heating (for example at 40-60° C.) optionally under vacuum, by placement under vacuum at room temperature (for example 20-25° C.), by freeze-drying).

Thus, the condensation step d2) will generally comprise:
a pre-condensation step d21) concomitant with the functionalization step c) and with the hydrolysis step d1) if necessary, during which part of the Si—OR¹⁰ groups and optionally of the SiOR⁴ groups will condense with one another; and
an advanced condensation step d22) after the functionalization step c) and the hydrolysis step d1) if necessary, during which more Si—OR¹⁰ groups and optionally SiOR⁴ groups will condense with one another.

During the advanced condensation step d22), the reaction medium is preferably dried or the pH is comprised between 6.8 and 7.8.

Thus, step d) is preferably carried out partly concomitantly with step c) then is favored by changing the reaction conditions as described above.

Step e)

Preferably, the process according to the invention further comprises a step e) of adding a molecule having the following formula Chem. III:

R⁷O—[R¹²R¹³SiO]ₚ—R⁸

or a salt thereof
in which:
p is an integer from 1 to 20;
R¹² and R¹³, which are identical or different, represent a hydrogen atom; a halogen atom; an —OR¹⁴ group with R¹⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl or a hydroxyl; and
R⁷ and R⁸, which are identical or different, represent a hydrogen atom, an aryl group or an aliphatic hydrocarbon group including from 1 to 6 carbon atoms.

Preferably, R¹² and R¹³, which are identical or different, represent an —OR¹⁴ group with R¹⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl or a hydroxyl.

In particular, R¹² and R¹³, which are identical or different, represent an —OR¹⁴ group with R¹⁴ representing a hydrogen atom, an aliphatic hydrocarbon group including from 1 to 6 carbon atoms, preferably a (C1-C6)alkyl group; or an aliphatic hydrocarbon group including from 1 to 6 carbon atoms, preferably a (C1-C6)alkyl group.

Advantageously, R⁷ and R⁸, which are identical or different, represent a hydrogen atom, or an aliphatic hydrocarbon group including from 1 to 6 carbon atoms, preferably a (C1-C6)alkyl group.

This molecule of formula Chem. III includes Si—OR groups (Si—OR⁷, Si—OR⁸ and optionally Si—OR¹⁴) capable of reacting with the Si—OR (Si—OR¹⁰ and optionally Si—OR⁴) groups of the molecule of formula Chem. I. Thus, during the sol-gel reaction allowing the formation of Si—O—Si bonds, a molecule of formula Chem. III can bind to two molecules of formula Chem. I grafted onto polysaccharide chains so as to form cross-linking bonds resulting from the coupling of a molecule of formula Chem. III with two molecules of formula Chem. I.

For example, the molecule of formula Chem. III is orthosilicic acid, tetraethyl orthosilicate (TEOS), polydimethylsiloxane (PDMS), oligomerized TEOS/orthosilicic acid, or methyl silanetriol (preferably used in the form of its sodium salt called sodium methyl siliconate—NaMS).

This step e) can be carried out after step d).

Preferably, step e) is carried out before or concomitantly with step d), in particular during step d21).

Step f)

Advantageously, the process according to the invention further comprises a step f) of adding an additional component selected from anesthetics, antioxidants, amino acids, vitamins, minerals, nucleic acids and mixtures thereof as described below.

Step g) Preferably, the process further comprises a purification step g), in particular by dialysis.

According to a particular embodiment, the purification is carried out after steps b) and c).

Preferably, the purification is carried out after steps b), c) and d).

More preferably, the purification is carried out after steps b), c), d1) and d21) and before step d22).

Step h)

Advantageously, the process further comprises a sterilization step h), in particular a heat sterilization step carried out at a plateau temperature comprised between 121° C. and 135° C., preferably for a plateau duration comprised between 1 minute and 20 minutes with FO 15, or a sterilization step by UV radiation. Preferably, the sterilization step is carried out after steps a) to d) and the optional steps e), f) and g).

Hydrogel

The present invention also relates to a hydrogel capable of being obtained by the process according to the present invention.

This hydrogel is preferably an injectable hydrogel. It is preferably sterile, in particular heat sterilized at a plateau temperature comprised between 121° C. and 135° C., preferably for a plateau duration comprised between 1 minute and 20 minutes with F0≥15.

This hydrogel is preferably homogeneous. This hydrogel is preferably stringy, with in particular a phase angle δ comprised between 20° and 45°. This hydrogel can also comprise an additional component selected from anesthetics, antioxidants, amino acids, vitamins, minerals, nucleic acids and mixtures thereof as described below.

The polysaccharide of this hydrogel is preferably as defined above, in the context of the description of step a) of the process according to the invention.

The cross-linking agent used to cross-link the polysaccharide of this hydrogel is preferably as defined above, in the context of the description of step b) of the process according to the invention. Preferably, it does not comprise units Si—O—Si, and in particular it does not comprise —[SiR²R³O]ₘ—SiR²R³— units.

The molecule Chem. I used to functionalize the polysaccharide of this hydrogel is preferably as defined above, in the context of the description of step c) of the process according to the invention.

The present invention also relates to a hydrogel comprising at least a polysaccharide cross-linked with at least one cross-linking bond LR1 and at least one cross-linking bond LR2,
the cross-linking bond LR1 comprising at least one unit Si—O—Si,
the cross-linking bond LR2, different from the cross-linking bond LR1, being obtained by cross-linking said polysaccharide with at least a cross-linking agent comprising at least two functional Z groups, which are identical or different, selected from isocyanate, amino, epoxide, carboxyl, N-succinimidyloxycarbonyl, N-sulfo succinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue, said hydrogel having a degree of modification by said at least a cross-linking agent of 0.05 to 10.0%, in particular from 0.05 to 5.0%, preferably from 0.1 to 1.0%.

This hydrogel is preferably an injectable hydrogel. It is preferably sterile, in particular sterilized by heat at a plateau temperature comprised between 121° C. and 135° C., preferably for a plateau duration comprised between 1 minute and 20 minutes with F0≥15. This hydrogel is preferably homogeneous. This hydrogel is preferably stringy, with in particular a phase angle δ comprised between 20° and 45°. This hydrogel can also comprise an additional component selected from anesthetics, antioxidants, amino acids, vitamins, minerals, nucleic acids and mixtures thereof as described below.

The polysaccharide of this hydrogel is preferably as defined above, in the context of the description of step a) of the process according to the invention.

The cross-linking agent used to cross-link the polysaccharide of this hydrogel is preferably as defined above, in the context of the description of step b) of the process according to the invention. Preferably, it does not comprise units Si—O—Si, and in particular it does not comprise —[SiR²R³O]ₘ—SiR²R³— units.

Thus, for example, when the cross-linking agent has the formula Chem. IIa above, the cross-linking bond LR2, obtained by cross-linking the polysaccharide with the cross-linking agent of formula Chem. IIa, will have the following formula Chem. V:

-G³-Y¹-G⁴-

in which:

Y¹ is as defined above, and

G³ and G⁴, which are identical or different, are binder groups bound to carbon atoms of the polysaccharide and resulting from the coupling of one of the functional Z¹ and Z² groups of the cross-linking agent of formula Chem. IIa with a functional group present on the polysaccharide (for example OH or COOH or else CHO for example when the polysaccharide is hyaluronic acid). G³ and G⁴, which are identical or different, are therefore for example selected from the following groups:

*—O—CO—NH—** (coupling of OH with an isocyanate), *—NH—CO—NH— (coupling of NH₂ with an isocyanate), *—CO—NH—** (coupling of COOH with an amino), *—O—CH₂—CH(OH)—** (coupling of OH with an epoxide), *—COO—CH₂—CH(OH)—** (coupling of COOH with an epoxide), *—O—CO—** (coupling of OH with a carboxyl, N-succinimidyloxycarbonyl, N-sulfosuccinimidyloxycarbonyl, or halogenocarbonyl), *—NH—CO—** (coupling of NH₂ with a carboxyl, N-succinimidyloxycarbonyl, N-sulfosuccinimidyloxycarbonyl, or halogenocarbonyl), *—O—CS—NH—** (coupling of OH with an isothiocyanate), *—NH—CS—NH— (coupling of NH₂ with an isothiocyanate), *—O—CH₂—CH₂—** (coupling of OH with a vinyl), *—O—CH(OH)—** (coupling of OH with a formyl), *—NH—CH(OH)—** (coupling of NH₂ with a formyl), *—CO—O—** (coupling of COOH with a hydroxyl), *—CO—S—** (coupling of COOH with a sulfhydryl), *═NH—NH—** (coupling of CHO with a hydrazino), *═NH—NH—CO—** (coupling of CHO with an acylhydrazino), *═NH—O—** (coupling of CHO with an aminoxy), *—CO—NR^{a}—CO—NH—** with R^{a} as defined above (coupling of COOH with a carbodiimide), *—O—CO—CH₂N(CH₂COOH)— (coupling of OH with the acid anhydride residue of formula Chem. GR3), *—NH—CO—CH₂N(CH₂COOH)— (coupling of NH₂ with the acid anhydride residue of formula Chem. GR3), *—O—CO—CH₂CH(COOH)—** (coupling of OH with the succinic anhydride residue), *—NH—CO—CH₂—CH(COOH)—** (coupling of NH₂ with the succinic anhydride residue), *—O—CO—CH═C(COOH)—** (coupling of OH with the maleic anhydride residue), *—NH—CO—CH═C(COOH)—** (coupling of NH₂ with the maleic anhydride residue), in which:

* represents the point of attachment to a carbon atom of the polysaccharide, and

** represents the point of attachment to Y¹.

The cross-linking bond LR1 will more particularly comprise a unit having the following formula Chem. IVa:

—Si(R⁵¹)(R⁶¹)—O—[SiR¹²¹R¹³¹O]ₚ₁—Si(R⁵²)(R⁶²)—

and more particularly will be a divalent group having the following formula Chem. IV:

-G′A¹-Si(R⁵¹)(R⁶¹)—O—[SiR¹²¹R¹³¹O]ₚ₁—Si(R⁵²)(R⁶²)-A²-G²-

in which:
p1 is an integer from 0 to 20, preferably equal to 0, A¹ and A², which are identical or different, represent a chemical bond or a spacer group, each corresponding in particular to a group A as defined above, within the context of the description of step c) of the process according to the invention,
R⁵¹, R⁵², R⁶¹ and R⁶², which are identical or different, represent a hydrogen atom; a halogen atom; an —OR⁴¹ group with R⁴¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more group(s) selected from a halogen atom, an aryl and a hydroxyl, R¹²¹ and R¹³¹, which are identical or different, represent a hydrogen atom; a halogen atom; an —OR¹⁴¹ group with R¹⁴¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from a halogen atom, an aryl or a hydroxyl, and
G¹ and G², which are identical or different, are binder groups bound to carbon atoms of the polysaccharide and selected from *—O—CO—NH—**. *—NH—CO—NH—. *—CO—NH—**. *—O—CH₂—CH(OH)—**, *—COO—CH₂—CH(OH)—**, *—O—CO—**, *—NH—CO—**, *—O—CS—NH—**, *—NH—CS—NH—, *—O—CH₂—CH₂—**, *—O—CH(OH)—**, *—NH—CH(OH)—**, *—CO—O—**, *—CO—S—**, *═NH—NH—**, *═NH—NH—CO—**, *═NH—O—**, *—CO—NR^{a}—CO—NH—** with R^{a} as defined above, *—O—CO—CH₂—N(CH₂—COOH)—**, *—NH—CO—CH₂N(CH₂—COOH)—**, *—O—CO—CH₂—CH(COOH)—**, *—NH—CO—CH₂—CH(COOH)—**, *—O—CO—CH═C(COOH)—** and *—NH—CO—CH═C(COOH)—**,
where * represents the point of attachment to a carbon atom of the polysaccharide, and
** represents the point of attachment to A¹ for G¹ and to A² for G².

Advantageously, R⁵¹, R⁵², R⁶¹ and R⁶², which are identical or different, represent an —OR⁴¹ group with R⁴¹ representing a hydrogen atom, an aryl group or an aliphatic hydrocarbon group including from 1 to 6 carbon atoms; or an aliphatic hydrocarbon group including from 1 to 6 carbon atoms optionally substituted by one or more groups selected from a halogen atom, an aryl and a hydroxyl.

Advantageously, R⁵¹, R⁵², R⁶¹ and R⁶², which are identical or different, represent an —OR⁴¹ group with R⁴¹ representing a hydrogen atom, an aryl group or an aliphatic hydrocarbon group including from 1 to 6 carbon atoms, preferably with R⁴¹ representing H or an aliphatic hydrocarbon group including from 1 to 6 carbon atoms, such as a (C1-C6)alkyl group.

Preferably:
p1 is as defined above and advantageously represents 0;
G¹ and G², which are identical or different, preferably identical, are as defined above and advantageously represent a *—CO—NH—** or *—O—CH₂—CH(OH)—** group, preferably an *—O—CH₂—CH(OH)—** group;
A¹ and A², which are identical or different, preferably identical, are each a divalent chain —(C1-C6)alkylene-O—((C1-C6)alkylene-, in particular —(C1-C4)alkylene-O—(C1-C4)alkylene-, such as —CH₂—O—(CH₂)₃—, the CH₂ group being preferably bound to G¹ or G² respectively and the (CH₂)₃ group being bound to Si; and
R⁵¹, R⁵², R⁶¹ and R⁶², which are identical or different, are each an —OR⁴¹ group with R⁴¹ representing H or a (C1-C6)alkyl group, preferably H, a methyl or an ethyl; or a (C1-C6)alkyl group, preferably a methyl or an ethyl; preferably R⁵¹, R⁵², R⁶¹ and R⁶², which are identical or different, are each an —OR⁴¹ group with R⁴¹ representing H or a (C1-C6)alkyl group, preferably H, a methyl or an ethyl.

The cross-linking bond LR1 advantageously results from the functionalization of the polysaccharide with a molecule of formula Chem. I as defined above and a sol-gel reaction allowing additional cross-linking, optionally in the presence of a molecule of formula Chem. III. Thus, in this case, the binder groups G¹ and G² result from the coupling of a functional group T of the molecule of formula Chem. I with a functional group present on the polysaccharide (for example OH or COOH or else CHO).

In particular, the hydrogel has a degree of modification by the molecule of formula Chem. I from 5 to 50%, preferably from 10 to 45%, in particular from 5 to 25%.

Composition

The present invention also relates to a composition comprising the hydrogel according to the present invention. It is preferably a cosmetic or pharmaceutical composition. It may also comprise physiologically acceptable excipients.

The hydrogel according to the invention comprises a cross-linked polysaccharide (for example, hyaluronic acid). The composition may also comprise a non-cross-linked polysaccharide (for example, hyaluronic acid).

The composition according to the present invention may thus comprise from 0.1 to 5% by weight, preferably from 1 to 3% by weight of polysaccharide (for example, hyaluronic acid), relative to the total weight of said composition, the polysaccharide (for example, hyaluronic acid) being present in cross-linked and optionally non-cross-linked form. In particular, the non-cross-linked polysaccharide (for example, hyaluronic acid) content varies from 0 to 40% by weight, preferably from 1 to 40% by weight, more preferably from 5 to 30% by weight, relative to the total weight of polysaccharide (for example, hyaluronic acid) present in the composition.

The polysaccharide of the hydrogel is preferably as defined above, in the context of the description of step a) of the process according to the invention, in particular hyaluronic acid.

The composition according to the present invention is preferably a sterile composition, in particular heat sterilized at a plateau temperature comprised between 121° C. and 135° C., preferably with a shelf life comprised between 1 minute and 20 minutes with F0≥15. It is preferably an injectable composition. The composition according to the invention then preferably comprises a physiologically acceptable medium, preferably a physiologically acceptable aqueous medium.

The physiologically acceptable aqueous medium can comprise a solvent or a mixture of physiologically acceptable solvents and preferably comprises water.

The physiologically acceptable medium can also comprise isotonic agents such as monosaccharides, sodium chloride and a mixture thereof.

The physiologically acceptable medium may further comprise at least one isotonic and physiologically acceptable saline solution.

Preferably, said balanced saline solution is a phosphate-buffered saline solution, and particularly a KH₂PO₄/K₂HPO₄ saline solution buffer. The composition according to the invention may further comprise at least an additional compound selected from anesthetics, antioxidants, amino acids, vitamins, minerals, nucleic acids, co-enzymes, adrenaline derivatives, and mixtures thereof.

As anesthetics mention may be made of Ambucaine, Amoxecaine, Amyleine, Aprindine, Aptocaine, Articaine, Benzocaine, Betoxycaine, Bupivacaine, Butacaine, Butamben, Butanilicaine, Chlorobutanol, Chloroprocaine, Cinchocaine, Clodacaine, Cocaine, Cryofluorane, Cyclomethycaine, Dexivacaine, Diamocaine, Diperodon, Dyclonine, Etidocaine, Euprocine, Febuverine, Fomocaine, Guafecainol, Heptacaine, Hexylcaine, Hydroxyprocaine, Hydroxytetracaine, Isobutamben, Leucinocaine, Levobupivacaine, Levoxadrol, Lidamidine, Lidocaine, Lotucaine, Menglytate, Mepivacaine, Meprylcaine, Myrtecaine, Octacaine, Octodrine, Oxetacaine, Oxybuprocaine, Parethoxycaine, Paridocaine, Phenacaine, Piperocaine, Piridocaine, Polidocanol, Pramocaine, Prilocaine, Procaine, Propanocaine, Propipocaine, Propoxycaine, Proxymetacaine, Pyrrocaine, Quatacaine, Quinisocaine, Risocaine, Rodocaine, Ropivacaine, Tetracaine, Tolycaine, Trimecaine or a salt thereof, in particular a hydrochloride thereof, and a mixture thereof.

As antioxidants, mention may be made of glutathione, reduced glutathione, ellagic acid, spermine, resveratrol, retinol, L-carnitine, polyols, polyphenols, flavonols, theaflavins, catechins, caffeine, ubiquinol, ubiquinone, alpha-lipoic acid and their derivatives, and a mixture thereof.

As amino acids, mention may be made of arginine (for example, L-arginine), isoleucine (for example, L-isoleucine), leucine (for example, L-leucine), lysine (for example, L-lysine or L-lysine monohydrate), glycine, valine (for example, L-valine), threonine (for example, L-threonine), proline (for example, L-proline), methionine, histidine, phenylalanine, tryptophan, cysteine, their derivatives (for example, N-acetyl derivatives such as N-acetyl-L-cysteine) and a mixture thereof.

As vitamins, and salts thereof, mention may be made of vitamins E, A, C, B, especially vitamins B6, B8, B4, B5, B9, B7, B12, and better still pyridoxine and derivatives and/or salts thereof, preferably pyridoxine hydrochloride.

As minerals, mention may in particular be made of zinc salts (for example, zinc acetate, which is in particular dehydrated), magnesium salts, calcium salts, potassium salts, manganese salts, sodium salts, copper salts (for example, copper sulfate, which is in particular pentahydrate), optionally in a hydrated form, and mixtures thereof.

As nucleic acids, particular mention may be made of adenosine, cytidine, guanosine, thymidine, cytodine, their derivatives and a mixture thereof.

As co-enzymes, mention may be made of coenzyme Q10, CoA, NAD, NADP, and mixtures thereof.

As adrenaline derivatives, mention may be made of adrenaline, noradrenaline and a mixture thereof.

The amounts of additional compounds of course depend on the nature of the compound in question, on the desired effect, and on the destination of the composition as described here.

Applications

The hydrogel or the composition according to the invention can have therapeutic, cosmetic and/or aesthetic applications.

The present invention therefore also relates to a hydrogel or a composition according to the invention for its use in filling and/or replacing tissues, in particular soft tissues, in particular by injecting the hydrogel or the composition into the tissue.

In particular, the hydrogel or the composition according to the invention is used in oral care and more particularly in the treatment of gingival recession, or to fill periodontal pockets. More particularly, the hydrogel or the composition according to the invention is used to treat the defects of the gingival architecture which can occur with tooth loss, with aging, with periodontal diseases and disorders, or after the installation of tooth implants, crowns or bridges.

The hydrogel or the composition according to the invention can also be used in ophthalmology, more particularly to protect the ocular structures during eye surgery such as for example ophthalmic surgery of the anterior or posterior segment, the extraction of the cataract possibly with implantation of an intraocular lens, corneal transplant surgery, filtering surgery for glaucoma, or implantation of a secondary lens.

In this case, the hydrogel or the composition according to the invention will be more particularly injected into the eye.

The hydrogel or the composition according to the invention can also be used in orthopedics or rheumatology, for example by injection into the synovial cavity. The hydrogel or the composition according to the invention is then used as viscosupplementation.

The hydrogel or the composition according to the invention can also be used in the treatment of lipodystrophy.

The hydrogel or the composition according to the invention can be used in cosmetic surgery, in particular for gynecoplasties and/or penoplasties.

The hydrogel or the composition according to the invention is administered more particularly by injection.

The present invention also relates to a process for treating the pathologies indicated above which comprises the administration, to an individual in need thereof, of an effective dose of the hydrogel or of the composition.

The effective dose of the hydrogel or of the composition varies according to many parameters such as, for example, the route of administration chosen, the weight, the age, the sex, the state of progress of the pathology to be treated and the sensitivity of the individual to be treated.

The present invention preferably relates to the aesthetic, and therefore non-therapeutic, use of a hydrogel or of a composition according to the invention for preventing and/or treating the alteration of the viscoelastic or biomechanical properties of the skin, and in particular for regenerating, moisturizing, firming or restoring the radiance of the skin, in particular by mesotherapy; to fill volume defects of the skin, and in particular to fill wrinkles, fine lines or scars (in particular hollow scars); or to reduce the appearance of fine lines and wrinkles.

For example, the present invention relates to the aesthetic use of a hydrogel or of a composition according to the invention for attenuating the nasolabial folds and bitterness folds; to increase the volume of the cheekbones, chin or lips; to restore the volumes of the face, in particular of the cheeks, the temples, the oval of the face, and around the eye; or to regenerate, hydrate, firm or restore the radiance of the skin, in particular by mesotherapy.

In particular, the hydrogel or the composition according to the invention is a hydrogel or an anti-aging composition. The hydrogel or the composition according to the invention is administered more particularly by injection.

The present invention also relates to a process for the cosmetic, preferably anti-aging, treatment of keratin materials, in particular the skin, comprising at least a step of administering a hydrogel or a composition according to the invention on or through said keratin materials, more particularly by injection.

The administration can be an injection, in particular an intraepidermal and/or intradermal and/or subcutaneous injection. Administration by intraepidermal and/or intradermal and/or subcutaneous injection according to the invention aims at injecting a hydrogel or a composition of the invention into an epidermal, dermo-epidermal and/or dermal region. The hydrogel or the composition according to the invention can also be administered by a supraperiosteal injection.

The hydrogel or the composition according to the invention can be injected using any of the processes known to the person skilled in the art. In particular, a hydrogel or a composition according to the invention can be administered by means of an injection device adapted for intraepidermal and/or intradermal and/or subcutaneous and/or supraperiosteal injection. The injection device can in particular be selected from a syringe, a set of microsyringes, a laser or hydraulic device, an injection gun, a needleless injection device, or a microneedle roller.

The injection device may include any injection means usually used suitable for intraepidermal and/or intradermal and/or subcutaneous and/or supraperiosteal injection. Preferably, such a means may be a hypodermic needle or a cannula.

A needle or cannula according to the invention may have a diameter varying from 18 to 34 G, preferably between 25 and 32 G, and a length varying from 4 to 70 mm, and preferably from 4 to 25 mm. The needle or cannula is advantageously disposable.

Advantageously, the needle or cannula is associated with a syringe or any other device allowing to deliver said hydrogel or said injectable composition through the needle or the cannula.

According to a variant embodiment, a catheter can be inserted between the needle/cannula and the syringe. In a known manner, the syringe can be actuated manually by the practitioner or else by a syringe support such as guns.

Preferably, the injection device can be selected from a syringe or a set of microsyringes.

In a variant embodiment, the injection device can be adapted to the mesotherapy technique.

Mesotherapy is a treatment technique by intraepidermal and/or intradermal and/or subcutaneous injection of a composition or a hydrogel. The composition or the hydrogel is administered according to this technique by injection in the form of multiple small droplets at the epidermis, the dermo-epidermal junction and/or the dermis in order, in particular, to produce a subcutaneous coating. The mesotherapy technique is described in particular in the work “Traité de mésothérapie” by Jacques LE COZ, Masson edition, 2004. Mesotherapy performed on the face is also called mesolift, or also under the term “mesoglow”.

The administration can also be topical.

Preferably, it is a topical application on the surface of the skin, more particularly on the epidermis, even more particularly on the facial epidermis.

The present invention is illustrated by the non-limiting examples below.

### EXAMPLES

Material
GPTMS: (3-Glycidyloxypropyl)trimethoxy-silane (Sigma 440167),
NaHA: non-cross-linked sodium hyaluronate 1.5 MDa and 4 MDa (HTL),
0.25M and 0.2M NaOH,
1M HCl (Chem Lab),
PBS Phosphate Buffer (Braun),
Lidocaine Hydrochloride,
Lyophilizer,
Three-Dimensional Shaker Turbula®,
Rheometer DHR-2,
Dynamometer Mecmesin AFG 100N,
Test Bench Mecmesin 2.5-dV,
Paddle homogenizer mill,
Sterile bag for paddle homogenizer mill.

Measurement of Viscoelastic Properties

The viscoelastic properties of the hydrogels obtained were measured using a rheometer (DHR-2) having a stainless steel cone (1°- 40 mm) with cone-plane geometry and an anodized aluminum peltier plate (42 mm) (air gap 24 μm). 0.5 g of sterilized hydrogel is deposited between the peltier plate and said cone. Then a stress scan is performed at 1 Hz and 25° C. The elastic modulus G′, the viscous modulus G″ and the phase angle δ are reported for a stress of 5 Pa.

The stress at the intersection of G′ and G″ τ is determined at the intersection of the curves of the modules G′ and G″ and is expressed in Pascal.

Cohesiveness Measurement

For the measurement of cohesiveness (or mechanical resistance, expressed in Newtons), the gel is deposited on the peltier plate with an initial air gap of 2.60 mm then left to rest for 60 seconds. The gel is then compressed at a constant speed of 100 μm/s up to 70% of the initial air gap, at 25° C. The cohesiveness of the gel is measured at the end of the compression stroke.

Measurement of the Extrusion Force

The extrusion forces (in Newtons) of the gels packaged in syringes were conducted through a test bench equipped with a dynamometer at a constant speed of 12.5 mm/min, through a 27 G ½″ needle and at room temperature. The extrusion force results correspond to the average of the average extrusion forces on at least 2 samples.

### Example 1: Preparation and Analyzes of Samples Based on BDDE and/or GPTMS-Modified Hyaluronic Acid

Prototypes no 1 to 12 are prepared as follows with the contents of NaOH, NaHA 1.5 MDa, BDDE, GPTMS described in Table 1:
Sodium hyaluronate (120 mg of NaHA per gram of reaction medium) and the BDDE are dissolved in a sodium hydroxide solution with a concentration according to Table 1 in a sterile bag;
The mixture is homogenized in a paddle mill for 2 cycles of 15 min at 210 rpm;
The GPTMS is added to the sterile bag;
The mixture is homogenized in a paddle mill for 2 cycles of 15 min at 210 rpm;
The mixture is maintained at 21° C. for 72 hours, the pH of the mixture is approximately 13;
A 1N HCl solution is added to the sterile bag until a pH of 7±0.5 is obtained;
The mixture is diluted to a concentration of 23 mg of HA/g of product with PBS phosphate buffer;
The mixture is homogenized for 24 hours using a three-dimensional shaker;
The mixture is dialyzed;
A sodium hyaluronate NaHA 4 MDa is added as a lubricant;
The aqueous solution of lidocaine hydrochloride is added to obtain 0.3% by weight of lidocaine hydrochloride relative to the weight of the resulting product;
The product thus obtained is sieved (710 μm sieve);
In the case of prototypes n° 11 and 12, the product is at this stage dried by lyophilization (freezing at −80° C. for 6 hours followed by vacuum drying for 48 hours) then rehydrated in 24 hours at a concentration of 23 mg/mL using deionized water;
The product thus obtained is packaged in a syringe;
Finally, the product is sterilized in an autoclave (plateau temperature comprised between 121° C. and 135° C. with FO 15).

Prototypes n° 2 and 12 are comparative.

Prototypes n° 1 and 3-11 are hydrogels according to the present invention.

The results are reported in Table 1 below.

| | | | Content | Cross-linking agent BDDE | | Functionalizing agent GPTMS | |
|---|---|---|---|---|---|---|---|
| | | NaOH | in HA | Cross-linking rate | | Functionalization rate | |
| | | concentration | (mol %/ | Mass | Molar | Mass | Molar |
| | | (mol/L) | NaOH) | rate (%) | rate (%) | rate (%) | rate (%) |
| Prototype | 1 | 0.25 | 12 | 3.0 | 6.2 | 20.1 | 42.8 |
| No | 2 | 0.25 | 12 | 0.0 | 0.0 | 6.7 | 12.2 |
| | 3 | 0.25 | 12 | 0.5 | 1.0 | 20.6 | 44.1 |
| | 4 | 0.25 | 12 | 1.1 | 2.2 | 6.7 | 12.3 |
| | 5 | 0.25 | 12 | 2.0 | 4.1 | 6.8 | 12.4 |
| | 6 | 0.25 | 12 | 3.0 | 6.2 | 6.7 | 12.3 |
| | 7 | 0.25 | 12 | 0.2 | 0.4 | 20.6 | 44.2 |
| | 8 | 0.20 | 15 | 0.2 | 0.4 | 15.9 | 32.1 |
| | 9 | 0.25 | 12 | 5.0 | 10.4 | 5.6 | 10.1 |
| | 10 | 0.25 | 12 | 0.2 | 0.4 | 20.6 | 44.2 |
| | 11 | 0.25 | 12 | 5.0 | 10.4 | 5.6 | 10.1 |
| | 12 | 0.25 | 12 | 1.1 | 2.1 | 0.0 | 0.0 |

After sterilization, the prototypes are analyzed, the elastic modulus G′, the phase angle δ and the stress at the intersection of G′ and G″ are determined. The results are reported in Table 2 below.

| | | | Stress | | | | |
|---|---|---|---|---|---|---|---|
| | | | at the | | | | |
| | | | intersection | | | MOD | MOD |
| Prototype | | | of G′ and | Extrusion | Cohesiveness | BDDE | GPTMS |
| No | G′ (Pa) | Delta (°) | G″ (Pa) | force (N) | (N) | (% ⁽²⁾) | (% ⁽¹⁾) |
| 1 | 594 ± 20 | 8.2 ± 0.3 | 270 ± 13 | 9.3 ± 0.2 | Not | 3.0 | 12.5 |
| | | | | | measured | | |
| 2 | 1 ± 0 | 76.1 ± 2.4 | ND | 5.0 ± 0.2 | Not | 0 | 5 |
| | | | | | measured | | |
| 3 | 424 ± 18 | 8.5 ± 0.5 | 404 ± 20 | 11.0 ± 0.4 | Not | 1.3 | 27.0 |
| | | | | | measured | | |
| 4 | 51 ± 2 | 34.3 ± 0.6 | 79 ± 3 | 11.4 ± 0.4 | 4.7 | 1.5 | 6 |
| 5 | 175 ± 9 | 15.8 ± 0.5 | 419 ± 3 | 11.4 ± 0.4 | 7.7 | 2.5 | 7 |
| 6 | 251 ± 17 | 10.1 ± 0.7 | 450 ± 9 | 9.6 ± 0.4 | Not | 5.3 | 5.5 |
| | | | | | measured | | |
| 7 | 319 ± 17 | 10.0 ± 0.5 | 418 ± 21 | 13.5 ± 0.7 | Not | 0.2 | 18 |
| | | | | | measured | | |
| 8 | 151 ± 8 | 22.4 ± 0.0 | 287 ± 3 | 15.9 ± 0.6 | 9.8 | Undetectable | 9 |
| 9 | 582 ± 28 | 6.0 ± 0.5 | 325 ± 50 | 9.2 ± 0.3 | Not | 8.5 | 5 |
| | | | | | measured | | |
| 10 | 1153 ± 96 | 9.0 ± 0.0 | 26 ± 0 | 6.4 ± 0.3 | Not | 0.2 | 18.0 |
| | | | | | measured | | |
| 11 | 1675 ± 147 | 11.2 ± 1.1 | 41 ± 4 | 6.5 ± 0.1 | Not | 8.5 | 5.0 |
| | | | | | measured | | |
| 12 | 15 ± 2 | 49.6 ± 6.7 | ND | 7.9 ± 0.4 | Not | 1.8 | 0.0 |
| | | | | | measured | | |

ND: Not Determinable
⁽¹⁾ % mol of GPTMS/100 moles of repeat units of HA
⁽²⁾ % mol of BDDE/100 moles of repeat units of HA

The control prototypes 2 and 12 without GPTMS or without BDDE do not allow to obtain gels with acceptable mechanical properties for the therapeutic, cosmetic and/or aesthetic applications targeted by the present invention. It is indeed the combination of BDDE and GPTMS which allows to obtain injectable elastic gels having the desired mechanical properties.

With mass contents of 1% BDDE and 6% GPTMS (prototype n° 4) (molar contents of 2% BDDE and 12% GPTMS), the prepared gel has desirable mechanical properties for the applications targeted in the scope of the present invention. Gels prepared with 0.2% by weight of BDDE (0.4% molar) and 15% or 20% by weight of GPTMS (32 and 44% molar) (respectively prototypes 8 and 7) have good rheological and injectability properties.

It should be noted that the prototype 8 is a gel with particularly advantageous, stringy sensory properties.

The effect of drying by freeze-drying then rehydration allows to exacerbate the elastic properties of the gels. Thus, the prototype 11, freeze-dried and rehydrated, shows a higher G′ than prototype 9 prepared with the same BDDE and GPTMS contents.

The same is observed when comparing prototypes 10 and 7.

### Example 2: Study of the Effect of NaMS

Prototype n° 10 is prepared as described in Example 1. Prototype 13 is prepared in the same way with, at the time of addition of GPTMS, the addition of NaMS in a ratio of 1:6 (mole of NaMS: mole of disaccharide unit of NaHA).

The NaOH, NaHA (1.5 MDa), BDDE and GPTMS contents are described in Table 3 below.

| | | | Cross-linking agent BDDE | | Functionalizing agent GPTMS | |
|---|---|---|---|---|---|---|
| | NaOH | HA content | Cross-linking rate | | Functionalization rate | |
| Prototype | concentration | (mol %/ | Mass | Molar | Mass | Molar |
| No | (mol/L) | NaOH) | rate (%) | rate (%) | rate (%) | rate (%) |
| 10 | 0.25 | 12 | 0.2 | 0.4 | 20.6 | 44.2 |
| 13 | 0.25 | 12 | 0.2 | 0.4 | 20.6 | 44.0 |

After sterilization, the prototypes are analyzed, the elastic modulus G′, the phase angle δ and the stress at the intersection of G′ and G″ are determined. The results are reported in Table 4 below.

| | | | Stress at the | |
|---|---|---|---|---|
| Prototype | | | intersection of | Extrusion |
| No | G′(Pa) | δ (°) | G′ and G″ (Pa) | force (N) |
| 10 | 1153 ± 96 | 9.0 ± 0.0 | 26 ± 0 | 6.4 ± 0.3 |
| 13 | 1843 ± 14 | 10.0 ± 0.4 | 64 ± 9 | 7.5 ± 0.8 |

The above results show that a product prepared in the presence of NaMS (prototype 13) has improved mechanical properties compared to a product prepared identically but without NaMS (prototype 10).

### Example 3: Preparation and Analyzes of Samples Based on DVS and/or GPTMS Modified-Hyaluronic Acid

Prototypes n° 14 and 15 are prepared as follows with the contents of NaOH, NaHA (1.5 MDa), DVS and GPTMS described in Table 5:
GPTMS, then NaOH then sodium hyaluronate NaHA (120 mg of NaHA per gram of reaction medium) are added to a sterile bag;
The mixture is homogenized in a paddle mill for 3 cycles of 15 min at 210 rpm and by manual palpation of the bag between each cycle;
The DVS is added in the bag;
The mixture is homogenized in a paddle mill for 15 min at 210 rpm;
The mixture is maintained at 21° C. for 48 hours, the pH of the mixture is approximately 13;
A 1M HCl solution is added to the sterile bag until a pH of 7±0.5 is obtained;
The mixture is diluted to a concentration of 23 mg of HA/g of product with PBS phosphate buffer;
The mixture is homogenized for 24 hours using a three-dimensional shaker,
The mixture is dialyzed;
A sodium hyaluronate NaHA 4 MDa is added as a lubricant;
The aqueous solution of lidocaine hydrochloride is added to the sterile bag to obtain 0.3% by weight of lidocaine hydrochloride relative to the weight of the resulting product;
The product thus obtained is sieved then packaged in a syringe;
Finally, the product is sterilized in an autoclave (plateau temperature comprised between 121° C. and 135° C. with FO 15).

The results are reported in Table 5 below.

| | |
|---|---|
| Cross-linking agent | Functionalizing agent |
| DVS | GPTMS |
| Cross-linking rate | Functionalization rate |

| Prototype | Mass rate | Molar rate | Mass rate | Molar rate |
|---|---|---|---|---|
| No | (%) | (%) | (%) | (%) |
| 14 | 0.4 | 1.4 | 0.0 | 0.0 |
| 15 | 0.3 | 1.0 | 20.6 | 44.2 |

After sterilization, the prototypes are analyzed, the elastic modulus G′, the phase angle δ and the stress at the intersection of G′ and G″ are determined. The results are reported in Table 6 below.

| | | | Stress at the | |
|---|---|---|---|---|
| Prototype | | | intersection of | Extrusion |
| No | G′(Pa) | δ (°) | G′ and G″ (Pa) | force (N) |
| 14 | 10 ± 4 | 55.6 ± 2.6 | ND* | 5.6 ± 0.2 |
| 15 | 615 ± 40 | 11.3 ± 0.5 | 242 ± 7 | 9.5 ± 0.2 |

*ND: Not Determinable: prototype 14 has a viscous predominance with a modulus G″ greater than the modulus G′ including for low stresses (confirmed by its value of δ greater than 45°); there is therefore no intersection of the curves of G′ and G″.

The small amount of DVS used for the modification of the hyaluronic acid for preparing the prototype 14 does not allow to lead to the formation of a gel with acceptable mechanical properties for the therapeutic, cosmetic and/or aesthetic applications targeted by the present invention.

In contrast, in the case of prototype 15, with the additional use of GPTMS, even with such a low amount of DVS, a desirable gel is formed.

### Example 4: Preparation and Analyzes of Samples Based on BDDE and APTES-Modified Hyaluronic Acid

Prototype n° 16 is prepared as follows with the NaOH, NaHA (1.5 MDa), BDDE, APTES, EDC and NHS contents described in Table 5:
NaOH then sodium hyaluronate (50 mg/g) are added to a pot;
The mixture is placed in the refrigerator (4±1° C.) and homogenized with a spatula for 5 minutes every hour for 4 hours;
The BDDE and the mixture is homogenized with a spatula for 5 minutes;
The mixture is maintained at 21° C. for 72 hours, the pH of the mixture is approximately 13;
A 1M HCl solution is added to the pot until an acid pH between 4.5 and 6.5 is obtained;
The EDC and the NHS are added to the pot and the mixture is homogenized for 5 minutes with a spatula then left to react at 21° C. for 30 minutes;
The APTES is then added drop by drop and the mixture is homogenized with a spatula then left for 15 hours at 21° C.;
The mixture is diluted to a concentration of 23 mg of HA/g of product with PBS phosphate buffer;
The mixture is homogenized for 24 hours using a three-dimensional shaker;
The mixture is dialyzed;
A sodium hyaluronate NaHA 4 MDa is added as a lubricant;
The aqueous solution of lidocaine hydrochloride is added to the sterile bag to obtain 0.3% by weight of lidocaine hydrochloride relative to the weight of the resulting product;
The product thus obtained is sieved then packaged in a syringe;
Finally, the product is sterilized in an autoclave (plateau temperature comprised between 121° C. and 135° C. with FO 15).

The results are reported in Table 7 below.

| | | | Cross-linking agent BDDE | | Functionalizing agent FIT | |
|---|---|---|---|---|---|---|
| | | | Cross-linking rate | | Functionalization rate | |
| Prototype | EDC/HA | NHS/HA | Mass | Molar | Mass | Molar |
| No | molar ratio | molar ration | ratio (%) | ratio (%) | ratio (%) | ratio (%) |
| 16 | 1.5 | 1 | 0.7 | 1.5 | 35.7 | 101.0 |

After sterilization, the prototypes are analyzed, the elastic modulus G′, the phase angle δ and the stress at the intersection of G′ and G″ are determined. The results are reported in Table 8 below.

| | | | Stress at the | |
|---|---|---|---|---|
| Prototype | | | intersection of | Extrusion |
| No | G′(Pa) | δ (°) | G′ and G″ (Pa) | force (N) |
| 16 | 1707 ± 44 | 7.2 ± 0.1 | 136 ± 11 | 8.2 ± 0.4 |

Despite the use of a small amount of BDDE, thanks to the additional use of APTES for the modification of hyaluronic acid according to the present invention, the prototype 16 is a gel with acceptable mechanical properties for the therapeutic, cosmetic and/or aesthetic applications covered by the present invention.

1. A process for preparing a hydrogel comprising the following steps:
a) providing at least a polysaccharide or a salt thereof;
b) cross-linking the polysaccharide in the presence of 0.05 to 10 mol % of at least a cross-linking agent, or a salt thereof, per 1 mole of repeat units of the polysaccharide, the cross-linking agent comprising at least two functional Z groups, which are identical or different, selected from the group consisting of isocyanate, amino, epoxide, carboxyl,
N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide, and an acid anhydride residue;
c) functionalizing the polysaccharide with at least a molecule of formula Chem. I: 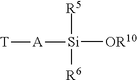
or a salt thereof
in which:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue;
A represents a chemical bond or a spacer group;
R⁵ and R⁶, which are identical or different, represent a hydrogen atom; a halogen atom; an —OR⁴ group with R⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more group(s) selected from the group consisting of a halogen atom, an aryl and a hydroxyl;
R¹⁰ represents a hydrogen atom, an aryl group or an aliphatic hydrocarbon group including from 1 to 6 carbon atoms;
d) sol-gel reacting at least a part of the Si—OR¹⁰ groups and optionally at least a part of the SiOR⁴ groups when they are present;
in which step b) is carried out before, or concomitantly with step c), or step b) is carried out consecutively to steps c) and d).

2. The process according to claim 1, wherein the polysaccharide is selected from the group consisting of pectin and pectic substances, chitosan, cellulose and derivatives thereof, agarose, glycosaminoglycans, heparosan, chondroitin sulfate, and mixtures thereof.

3. The process according to claim 1 wherein the polysaccharide is hyaluronic acid.

4. The process according to claim 1, wherein the cross-linking agent is a compound of formula Chem. II:
Y—(Z)ₙ
in which the Z groups, which are identical or different, are selected from the group consisting of isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide, and an acid anhydride residue;
n is an integer greater than or equal to 2;
Y is a polyvalent hydrocarbon group having a valence of n and including from 1 to 50 carbon atoms:
in which one or more units CH2 are optionally replaced by one or more divalent units selected from the group consisting of arylenes, —O—, —S—, —S(O)—, —C(═O)—, —SO₂—, —N(R¹)—, and —[SiR²R³O]ₘ—SiR²R³—, with
R¹ representing a hydrogen atom, an aliphatic hydrocarbon group including from 1 to 6 carbon atoms, or an aryl —(C1-C6)alkyl,
m being an integer comprised between 1 and 20, and
R² and R³, which are identical or different, representing a hydrogen atom; a halogen atom; an —OR¹¹ group with R¹¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from the group consisting of a halogen atom, an aryl and a hydroxyl;
said polyvalent hydrocarbon group being unsubstituted or substituted by one or more monovalent groups selected from the group consisting of a halogen atom, a hydroxyl and an aryl-(C1-C6)alkyl.

5. The process according to claim 1, wherein the cross-linking agent is a compound of formula Chem. IIa:
Z¹—Y¹—Z²
in which Z¹ and Z², which are identical or different, are selected from the group consisting of isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue, and
Y¹ represents a divalent aliphatic hydrocarbon chain including from 1 to 50 carbon atoms:
in which one or more units CH2 are optionally replaced by one or more divalent units selected from the group consisting of —O—, —SO₂—, —NH—, and —[SiR²R³O]ₘ—SiR²R³—, with m an integer comprised between 2 and 20, and
R² and R³, which are identical or different, representing a hydrogen atom; a halogen atom; an —OR¹¹ group with R¹¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from the group consisting of a halogen atom, an aryl and a hydroxyl;
said chain being unsubstituted or substituted by one or more monovalent groups selected from the group consisting of a halogen atom, a hydroxyl and an aryl-(C1-C6)alkyl.

6. The process according to claim 1 wherein the functional Z or Z¹ and Z² groups are identical and represent an epoxide group.

7. The process according to claim 1 wherein the cross-linking agent is selected from the group consisting of 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and mixtures thereof.

8. The process according to claim 1, wherein the functional Z or Z¹ and Z² groups are identical and selected from the group consisting of amino, vinyl, formyl and carbodiimide groups.

9. The process according to claim 1 wherein A is a divalent aliphatic hydrocarbon chain including from 1 to 12 carbon atoms:
in which are optionally interposed, between two carbon atoms of said chain, one or more divalent units selected from the group consisting of arylenes, —O—, —S—, —S(O)—, —C(═O)—, —SO₂— and —N(R⁹)— with R⁹ representing a hydrogen atom, an aliphatic hydrocarbon group including from 1 to 6 carbon atoms, or an aryl-(C1-C6)alkyl;
said chain being unsubstituted or substituted by one or more monovalent groups selected from the group consisting of a halogen atom, a hydroxyl and an aryl-(C1-C6)alkyl.

10. The process according to claim 1 wherein in the molecule of formula Chem. I:
T is an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide group, or an acid anhydride residue;
A is a divalent chain —(C1-C6)alkylene-O—(C1-C6)alkylene;
R⁵ and R⁶, which are identical or different, are each an —OR⁴ group with R⁴ representing a (C1-C6)alkyl group; or a (C1-C6)alkyl group and
R¹⁰ is a (C1-C6)alkyl group.

11. The process according to claim 1, wherein in step c), the polysaccharide is functionalized in the presence of 5 to 50 mol % of molecule of formula Chem. I or a salt thereof per 1 mole repeat unit of the polysaccharide.

12. The process according to claim 1, wherein steps b) and c) are concomitant.

13. The process according to claim 1, that further comprises a step e) of adding a molecule of formula Chem. III:
R⁷O—[R¹²R¹³SiO]ₚ—R⁸
or a salt thereof
in which:
p is an integer from 1 to 20;
R¹² and R¹³, which are identical or different, represent a hydrogen atom; a halogen atom; an —OR¹⁴ group with R¹⁴ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from the group consisting of a halogen atom, an aryl and a hydroxyl; and
R⁷ and R⁸, which are identical or different, represent a hydrogen atom, an aryl group or an aliphatic hydrocarbon group including from 1 to 6 carbon atoms.

14. The process according to claim 1, that further comprises a step f) of adding an additional component selected from the group consisting of anesthetics, antioxidants, amino acids, vitamins, minerals, nucleic acids and mixtures thereof.

15. A hydrogel obtainable according to the process of claim 1.

16. A hydrogel comprising at least a polysaccharide cross-linked with at least one cross-linking bond LR1 and at least one cross-linking bond LR2,
the cross-linking bond LR1 comprising at least one unit Si—O—Si,
the cross-linking bond LR2, different from the cross-linking bond LR1, being obtained by cross-linking a polysaccharide with at least a cross-linking agent comprising at least two functional Z groups, which are identical or different, selected from the group consisting of isocyanate, amino, epoxide, carboxyl, N-succinimidyloxycarbonyl, N-sulfo succinimidyloxycarbonyl, halogenocarbonyl, isothiocyanate, vinyl, formyl, hydroxyl, sulfhydryl, hydrazino, acylhydrazino, aminoxy, carbodiimide groups, and an acid anhydride residue;
the hydrogel having a degree of modification by the cross-linking agent of 0.05 to 10.0%.

17. The hydrogel according to claim 16, wherein the polysaccharide(s) is selected from the group consisting of pectin and pectic substances, chitosan, cellulose and derivatives thereof, agarose, glycosaminoglycans, heparosan or chondroitin sulfate, and mixtures thereof.

18. The hydrogel according to claim 16, wherein the cross-linking agent is a compound of formula Chem. II:
Y—(Z)ₙ
in which the Z groups, which are identical or different, are selected from the group consisting of isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide, and an acid anhydride residue;
n is an integer greater than or equal to 2:
Y is a polyvalent hydrocarbon group having a valence of n and including from 1 to 50 carbon atoms:
in which one or more units CH₂ are optionally replaced by one or more divalent units selected from the group consisting of arylenes, —O—, —S—, —S(O)—, —C(═O)—, —SO₂—, —N(R¹)—, and —[SiR²R³O]ₘ—SiR²R³—, with
R¹ representing a hydrogen atom, an aliphatic hydrocarbon group including from 1 to 6 carbon atoms, or an aryl —(C1-C6)alkyl,
m an integer comprised between 1 and 20, and
R² and R³, which are identical or different, representing a hydrogen atom: a halogen atom: an —OR¹¹ group with R¹¹ representing a hydrogen atom, an aryl group or an aliphatic hydrocarbon group including from 1 to 6 carbon atoms: an aryl: or an aliphatic hydrocarbon group including from 1 to 6 carbon atoms optionally substituted by one or more groups selected from the group consisting of a halogen atom, an aryl and a hydroxyl;
said polyvalent hydrocarbon group being unsubstituted or substituted by one or more monovalent groups selected from the group consisting of a halogen atom, a hydroxyl and an aryl-(C1-C6)alkyl.

19. The hydrogel according to claim 16 wherein the cross-linking bond LR1 is a divalent group having the following formula Chem. IV:
-G¹-A¹-Si(R⁵¹)(R⁶¹)—O—[SiR¹²¹R¹³¹O]ₚ₁—Si(R⁵²)(R⁶²)-A²-G²-
in which:
p1 is an integer from 0 to 20, A¹ and A², which are identical or different, represent a chemical bond or a spacer group, R⁵¹, R⁵², R⁶¹ and R⁶², which are identical or different, represent a hydrogen atom; a halogen atom; an —OR⁴¹ group with R⁴¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more group(s) selected from the group consisting of a halogen atom, an aryl and a hydroxyl,
R¹²¹ and R¹³¹, which are identical or different, represent a hydrogen atom; a halogen atom; an —OR¹⁴¹ group with R¹⁴¹ representing a hydrogen atom, an aryl group or an aliphatic hydrocarbon group including from 1 to 6 carbon atoms; an aryl; or an aliphatic hydrocarbon group including from 1 to 6 carbon atoms optionally substituted by one or more groups selected from the group consisting of a halogen atom, an aryl and a hydroxyl, and
G¹ and G², which are identical or different, are binder groups bound to carbon atoms of the polysaccharide(s) and selected from the group consisting of *—O—CO—NH—**, *—NH—CO—NH—, *—CO—NH—**, *—O—CH₂—CH(OH)—**, *—COO—CH₂—CH(OH)—**, *—O—CO—**, *—NH—CO—**, *—O—CS—NH—**, *—NH—CS—NH—, *—O—CH₂—CH₂—**, *—O—CH(OH)—**, *—NH—CH(OH)—**, *—CO—O—**, *—CO—S—**, *═NH—NH—**, *═NH—NH—CO—**, *═NH—O—**, *—CO—NR^{a}—CO—NH—**, *—O—CO—CH₂—N(CH₂—COOH)—**, *—NH—CO—CH₂—N(CH₂—COOH)—**, *—O—CO—CH₂—CH(COOH)—**, *—NH—CO—CH₂—CH(COOH)—**, *—O—CO—CH═C(COOH)—** and *—NH—CO—CH═C(COOH)—**, where R^{a} represents an aliphatic hydrocarbon group including from 1 to 20 carbon atoms, one or more carbon atoms of which are optionally replaced by a heteroatom selected from the group consisting of O, S and N,
* represents the point of attachment to a carbon atom of the polysaccharide, and
** represents the point of attachment to A¹ for G¹ and to A² for G².

20. The hydrogel according to claim 19, wherein A^{t} and A², which are identical or different, are each a divalent aliphatic hydrocarbon chain including from 1 to 12 carbon atoms:
in which are optionally interposed, between two carbon atoms of said chain, one or more divalent units selected from the group consisting of arylenes, —O—, —S—, —S(O)—, —C(═O)—, —SO₂— and —N(R⁹)— with R⁹ representing a hydrogen atom, an aliphatic hydrocarbon group including from 1 to 6 carbon atoms, or an aryl-(C1-C6)alkyl;
said chain being unsubstituted or substituted by one or more monovalent groups selected from the group consisting of a halogen atom, a hydroxyl and; an aryl-(C1-C6)alkyl.

21. The hydrogel according to claim 19 wherein:
G¹ and G², which are identical or different, are binder groups bound to carbon atoms of the polysaccharide(s) and selected from the group consisting of *—O—CO—NH—**, *—NH—CO—NH—, *—CO—NH—**, *—O—CH₂—CH(OH)—**, *—COO—CH₂—CH(OH)—**, *—O—CO—**, *—NH—CO—**, *—O—CS—NH—**, *—NH—CS—NH—, *—O—CH₂—CH₂—**, *—O—CH(OH)—**, *—NH—CH(OH)—**, *—CO—O—** *—CO—S—**, *═NH—NH—**, *═NH—NH—CO—**, *═NH—O—**, *—CO—NR^{a}—CO—NH—**, *—O—CO—CH₂—N(CH₂—COOH)—**, *—NH—CO—CH₂—N(CH₂—COOH)—**, *—O—CO—CH₂—CH(COOH)—**, *—NH—CO—CH₂—CH(COOH)—**, *—O—CO—CH═C(COOH)—** and *—NH—CO—CH═C(COOH)—**,
A¹ and A² represent, independently of each other, a divalent chain —(C1-C6)alkylene-O—(C1-C6)alkylene; and
R⁵¹, R⁵², R⁶¹ and R⁶² represent, independently of each other, an —OR⁴¹ group with R⁴¹ representing H or a (C1-C6)alkyl group; or a (C1-C6)alkyl group.

22. The hydrogel according to claim 16, that further comprises an additional component selected from the group consisting of anesthetics, antioxidants, amino acids, vitamins, minerals, nucleic acids and mixtures thereof.

23. A cosmetic or pharmaceutical composition comprising a hydrogel according to claim 16.

24. (canceled)

25. An aesthetic method for preventing and/or treating the alteration of the viscoelastic or biomechanical properties of the skin; to fill volume defects of the skin, to reduce the appearance of fine lines and wrinkles; or to regenerate, hydrate, firm or restore the radiance of the skin, that comprises administering to a subject a hydrogel according to claim 16.

26. The hydrogel according to claim 16, wherein the polysaccharide(s) is hyaluronic acid.

27. An aesthetic method for preventing and/or treating the alteration of the viscoelastic or biomechanical properties of the skin; to fill volume defects of the skin, to reduce the appearance of fine lines and wrinkles; or to regenerate, hydrate, firm or restore the radiance of the skin, that comprises administering to a subject a composition according to claim 23.
